SEQUENCE LISTING (1 sequence)

SEQ ID NO: 1 - protein, 9 aa, artificial
  synthetic
RPPGFSPFR

(19) United States
(12) Patent Application Publication
(10) Pub. No.: US 2005/0153940 A1
(43) Pub. Date: Jul. 14, 2005

(54) Method for using potassium channel activation for delivering a medicant to an abnormal brain region and/or a malignant tumor

(75) Inventors: Keith L. Black, Los Angeles, CA (US); Nagendra S. Ningaraj, Culver City, CA (US)

Correspondence Address: KING & SPALDING LLP, 191 PEACHTREE STREET, N.E., 45TH FLOOR, ATLANTA, GA, 30303-1763, US

(73) Assignee: Cedars-Sinai Medical Center

(21) Appl. No.: 10/998,866

(22) Filed: Nov. 29, 2004

Related U.S. Application Data
(63) Continuation of application No. 09/615,854, filed on Jul. 14, 2000, now abandoned.
Application No. 09/615,854 is a continuation-in-part of application No. 09/491,500, filed on Jan. 26, 2000.

(51) Int. Cl. A61K 31/655; A61K 31/5377

(52) U.S. Cl. 514/149; 514/235.5; 514/509

(57) ABSTRACT

Disclosed are methods of selectively delivering a medicant to an abnormal brain region and/or to a malignant tumor in a mammalian subject, including a human. A medicant is administered simultaneously or substantially simultaneously with a potassium channel activator (other than bradykinin or a bradykinin analog), such as an activator of soluble guanylyl cyclase (e.g., nitric oxide or a nitric oxide donor) or an activator of cyclic GMP-dependent protein kinase, whereby the medicant is delivered selectively to the cells of the abnormal brain region and/or to the tumor, compared to normal tissues. Thus, among the disclosures is a method of treating a malignant tumor in a human subject. Also disclosed are pharmaceutical compositions that combine a potassium channel activator together with a medicant and a kit for enhancing the delivery of a medicant to an abnormal brain region and/or to a malignant tumor.

This application is a continuation-in-part of U.S. Ser. No. 09/491,500, filed Jan. 26, 2000

### BACKGROUND OF THE INVENTION

Throughout the application various publications are referenced in parentheses. The disclosures of these publications in their entireties are hereby incorporated by reference in the application in order to more fully describe the state of the art to which this invention pertains.

1. The Field of the Invention

This invention relates to the medical arts. In particular, it relates to a method of enhancing the delivery of a medicant across abnormal microvasculature to a tissue requiring treatment.

2. Discussion of the Related Art

Pathologic neovascularization, i.e., the proliferation or development of new blood vessels, is essential for the growth and spread of primary, secondary and metastatic malignant tumors. It is known that certain properties of the new capillaries and arterioles constituting the neomicrovasculature in solid tumors differ from those of normal microvasculature. (J. Denekamp et al., Vasculature and microenvironmental gradients: the missing links in novel approaches to cancer therapy?, Adv. Enzyme Regul. 38:281-99 [1998]). Neomicrovasculature induced by angiogenic factors from malignant cells was reported to possess altered pharmacological reactivity to some vasoconstricting agents, compared with neomicrovasculature that was not induced by neoplastic cells. (S. P. Andrade and W. T. Beraldo, Pharmacological reactivity of neoplastic and non-neoplastic associated neovasculature to vasoconstrictors, Int. J. Exp. Pathol. 79(6): 425-32 [1998]).

A number of proposed cancer treatments have been based on differences between neomicrovasculature and normal microvasculature. For example, combretastatin A-4 was shown to cause vascular damage and occlusion selectively in the blood vessels of malignant tumors compared to normal blood vessels. (G. G. Dark et al., Combretastatin A-4, an agent that displays potent and selective toxicity toward tumor vasculature, Cancer Res. 57(10): 1829-34 [1997]; D. J. Chaplin et al., Anti-vascular approaches to solid tumour therapy: evaluation of combretastatin A4 phosphate, Anticancer Res. 19(1A): 189-95 [1999]). Monoclonal antibodies have been directed to antigens and antigenic combinations specific to endothelial cells of pathologic neovasculature, such as vascular cell adhesion molecule (VCAM)-1, phosphatidylserine (PS), the glycoprotein endosialin, and prostate-specific membrane antigen (PSMA), with the aim of selectively inducing thrombosis in neovasculature. (E.g., S. Ran et al., Infarcation of solid Hodgkins tumors in mice by antibody-directed targeting of tissue factor to tumor vasculature, Cancer Res. 58(20): 4646-53 [1998]; I. Ohizumi et al., Antibody-based therapy targeting tumor vascular endothelial cells suppresses solid tumor growth in rats, Biochem. Biophys. Res. Commun. 236(2): 493-96 [1997]; S. S. Chang et al., Five different antiprostate-specific membrane antigen (PSMA) antibodies confirm PSMA expression in tumor-associated neovasculature, Cancer Res. 59(13): 3192-98 [1999]; W. J. Rettig et al., Identification of endosialin, a cell surface glycoprotein of vascular endothelial cells in human cancer, Proc. Natl. Acad. Sci. USA 89(22): 10832-36 [1992]). But taken alone, shutting down blood flow through the neomicrovasculature to malignant tumors may not necessarily result in stopping tumor growth, because actively proliferating populations of neoplastic cells at the periphery of solid tumors may have access to blood supplied by normal microvasculature. (E.g., D. J. Chaplin et al. [1999]).

Consequently, other conventional and novel therapeutic modalities will continue to be of value in the treatment of malignant, solid tumors. However, the efficacy of novel therapeutic agents, including cytotoxic chemotherapeutic agents, monoclonal antibodies, cytokines, effector cells, and viral particles has been limited by their ability to reach their targets in vivo in adequate quantities. (E.g., R. K Jain, Vascular and interstitial barriers to delivery of therapeutic agents in tumors, Cancer Metastasis Rev. 9(3): 253-66 [1990]). An important limiting factor is the low permeability to macromolecules and viral particles of neomicrovasculature supplying the tumors.

This problem of microvascular permeability is especially acute with respect to malignant tumors of the central nervous system. These malignancies are usually fatal, despite recent advances in the areas of neurosurgical techniques, chemotherapy and radiotherapy. In particular, there are no standard therapeutic modalities that can substantially alter the prognosis for patients with malignant tumors of the brain, cranium, and spinal cord. For example, high mortality rates persist for patients diagnosed with malignant medulloblastomas, malignant meningiomas, malignant neurofibrosarcomas and malignant gliomas, which are characterized by infiltrative tumor cells throughout the brain. Although intracranial tumor masses can be debulked surgically, treated with palliative radiation therapy and chemotherapy, the survival associated with intracranial tumors, for example, a glioblastoma, is typically measured in months. The development of new therapeutic modalities against solid brain tumors largely depends on transvascular delivery of the potential therapeutic agent.

Transvascular delivery of chemotherapeutic agents and viral particles to tumor cells or other abnormal brain tissue is hampered by the blood-brain barrier, particularly the blood-tumor barrier found in brain tumors. The blood-brain barrier is a transvascular permeability barrier thought to result from the interendothelial tight junctions formed by the cerebrovascular endothelial cells of brain capillaries and arterioles in both normal and abnormal brain tissue; the maintenance of the blood-brain barrier possibly involves endogenous nitric oxide production and a cyclic GMP-dependent mechanism. (Liu, S. M. and Sundqvist, T., Nitric oxide and cGMP regulate endothelial permeability and F-actin distribution in hydrogen peroxide-treated endothelial cells, Exp. Cell. Res. 235(1): 238-44 [1997]). The blood-brain barrier protects the brain from changes in the composition of the systemic blood supply (e.g., in electrolytes) or from blood-borne macromolecules, such as immunoglobulins or other polypeptides, and prevents the transvascular delivery of many exogenously supplied pharmaceutical agents to brain tissues.

The treatment of brain tissue abnormalities, such as tumors, often involves the use of pharmaceutical agents with a significant toxicity of their own, making it highly desirable to be able to preferentially direct such agents to the abnormal or malignant tissue. While, there has been a great deal of interest in developing techniques which are capable of opening the blood-brain barrier to allow transport of pharmaceutical agents to the brain. Few of these methods are capable of selectively opening the blood-brain barrier only in the abnormal brain tissue while leaving the blood-brain barrier in the normal brain tissue intact.

For example, Neuwelt et al. used an intracarotid injection of hypertonic mannitol to osmotically disrupt the blood-brain barrier. They reported that this enhanced the uptake by brain tissue of inactivated HSV-1 particles that were administered immediately afterward by intracarotid bolus injection. (E. A. Neuwelt et al., Delivery of ultraviolet-inactivated 35S-herpesvirus across an osmotically modified blood-brain barrier, J. Neurosurg. 74(3): 475-79 [1991]; Also, S. E. Doran et al., Gene expression from recombinant viral vectors in the central nervous system after blood-brain barrier disruption, Neurosurgery 36(5): 965-70 [1995]; G. Nilayer et al., Delivery of herpesvirus and adenovirus to nude rat intracerebral tumors after osmotic blood-brain barrier disruption, Proc. Natl. Acad. Sci. USA 92(21): 9829-33 [1995]).

Intracarotid infusion of leukotriene C.sub.4 (LTC.sub.4) selectively increases the permeability in brain tumor capillaries without affecting the permeability in normal brain capillaries. The effect of LTC.sub.4 on brain tumor capillaries is, however, limited to small molecules and it can only slightly increase the permeability of those small molecules in abnormal brain tissue relative to normal. Accordingly, LTC.sub.4 does not significantly increase the delivery of some larger water soluble molecules to brain tumors or other abnormalities.

The vasoactive nanopeptide bradykinin and agonists or polypeptide analogs thereof (e.g., receptor-mediated permeabilizers [RMPs]) have been injected intravenously to increase blood-brain barrier permeability to co-administered neuropharmaceutical or diagnostic agents. (B. Malfroy-Camine, Method for increasing blood-brain barrier permeability by administering a bradykinin agonist of blood-brain barrier permeability, U.S. Pat. No. 5,112,596; J. W. Kozarich et al., Increasing blood brain barrier permeability with permeabilizer peptides, U.S. Pat. No. 5,268,164). Intracarotid infusion of bradykinin will selectively increase permeability 2- to 12-fold in brain tumor and ischemic brain capillaries for molecules ranging in molecular weight from 100 to 70,000 Daltons (Inamura, T. et al., Bradykinin selectively opens blood-tumor barrier in experimental brain tumors, J. Cereb. Blood Flow Metab. 14(5): 862-70 [1994]). Bradykinin does not increase permeability in the normal blood brain barrier except at very high doses. (Wirth, K. et al., DesArg9-D-Arg[Hyp3,Thi5,D-Tic7,Oic8]bradykinin (desArg10-[Hoe140]) is a potent bradykinin B1 receptor antagonist, Eur. J. Pharmacol. 205(2): 217-18 [1991]). Opening of the blood-tumor barrier by bradykinin is transient, lasting 15 to 20 minutes. (Inamura et al. [1994]). After opening of abnormal brain capillaries with bradykinin, the capillaries become refractory to the bradykinin effect for up to 60 minutes. (Inamura et al. [1994]).

A method for selectively delivering to abnormal brain tissue a neuropharmaceutical agent (e.g., 5-fluorouracil, cisplatin, methotrexate, or monoclonal antibodies) or a diagnostic agent (e.g., technicium-99 glucoheptonate, gallium-EDTA, and ferrous magnetic or iodinated contrasting agents) employed intracarotid infusion of bradykinin, or a bradykinin analog, such as RMP-7; the bradykinin or bradykinin analog was administered approximately contemporaneously with the agent. (K. L. Black, Method for selective opening of abnormal brain tissue capillaries, U.S. Pat. Nos. 5,527,778 and 5,434,137). Enhanced transvascular delivery of HSV-derived viral particles to malignant cells in the brains of rats was also achieved by disrupting the blood-brain barrier with bradykinin or RMP-7. (N. G. Rainov, Selective uptake of viral and monocrystalline particles delivered intra-arterially to experimental brain neoplasms, Hum. Gene. Ther. 6(12): 1543-52 [1995]; N. G. Rainov et al., Long-term survival in a rodent brain tumor model by bradykinin-enhanced intra-arterial delivery of a therapeutic herpes simplex virus vector, Cancer Gene Ther. 5(3): 158-62 [19981; F. H. Barnett et al., Selective delivery of herpes virus vectors to experimental brain tumors using RMP-7, Cancer Gene Ther. 6(1): 14-20 [1999]).

The calcium-activated potassium channel (K_{Ca}) is an important regulator of blood vessel tone (Nelson M T, Quayle J M. Physiological roles and properties of potassium channels in arterial smooth muscle, Am. J. Physiol. 268(4 Pt 1): C799-822[1995]; Bang, L. et al., Nitroglycerin-mediated vasorelaxation is modulated by endothelial calcium-activated potassium channels, Cardiovasc. Res. 43(3): 772-78 [1999]). The KCa channel is ubiquitously distributed in tissues as and subunits. Its activity is triggered by depolarization and enhanced by an increase in cytosolic calcium di-cation (Ca²⁺). A local increase in Ca²⁺ is sensed by the extremely sensitive brain-subunit of the K_{Ca}, directed towards the cytoplasm in the cell, that allows a significant potassium cation flux through these channels. Under conditions when intracellular cyclic 3′,5′ adenosine monophosphate (cAMP) concentration rises in vascular endothelium (e.g. hypoxia), ATP-sensitive potassium channels (K_{ATP}) may also play a role. (J. E. Brian et al., Recent insights into the regulation of cerebral circulation, Clin. Exp. Pharmacol. Physiol. 23(6-7): 449-57 [1996]). Minoxidil sulfate and chromakalim are reported to be activators of K_{ATP}. (A. D. Wickenden et al., Comparison of the effects of the K(+)-channel openers cromakalim and minoxidil sulphate on vascular smooth muscle, Br. J. Pharmacol, 103(1): 1148-52 [1991]).

Intimately connected with the regulation of potassium channels is guanosine 3′,5′-cyclic monophosphate, commonly known as cyclic GMP (cGMP), an important signal transducing molecule, which mediates the regulation of three main classes of effector proteins: (1) cGMP-dependent protein kinases, which mediate protein phosphorylation; (2) cGMP-gated ion channel protein kinases, which mediate cation influx across the plasma membrane; and (3) phosphodiesterases, which mediate cyclic nucleotide catabolism. (Lohse, M. J. et al., Pharamacology of NO:cGMPsignal transduction, Naunyn-Schmiedebergs Arch. Pharmacol.358:111-12 [1998]; Smolenski, A. et al., Functional analysis of cGMP-dependent protein kinases I and II as mediators of NO/cGMP effects, Naunyn-Schmiedebergs Arch. Pharmacol.358:134-39 [1998]; He, P. et al., cGMP modulates basal and activated microvessel permeability independently of [Ca2⁺]i, Am. J. Physiol. 274(6 Pt 2):H1865-74 [1998]; Holschermann, H. et al., Dual role of cGMP in modulation of macromolecule permeability of aortic endothelial cells, Am. J. Physiol. 272(1 Pt 2):H91-98 [1997]).

The production of cGMP from GTP is catalyzed by soluble guanylyl cyclase, a nitric oxide-activated enzyme. (Patel, A. I. and Diamond, J., Activation of guanosine 3′,5′-cyclic monophosphate (cGMP)-dependent protein kinase in rabbit aorta by nitroglycerin and sodium nitroprusside, J. Pharmacol. Exp. Ther. 283(2): 885-93 [1997]; Patel, A. I. et al., Activation of guanosine 3′,5′-cyclic monophosphate (cGMP)-dependent protein kinase in rat vas deferens and distal colon is not accompanied by inhibition of contraction, J. Pharmacol. Exp. Ther. 283(2): 894-900 [1997]).

There is also evidence that nitric oxide participates in the regulation of microvascular tone. (Joo, F. et al., Regulation of the macromolecular transport in the brain microvessels: the role of cyclic GMP, Brain Res.278(1-2): 165-74 1983]). For example, glial tumors and ischemic tissue are more immunopositive for NNOS and eNOS relative to normal brain. (Cai, Z. et al., Prenatal hypoxia-ischemia alters expression and activity of nitric oxide synthase in young rat brain and causes learning deficits, Brain Res. Bull. 49(5): 359-65 [1999]; Nakano, S. et al., Increased brain tumor microvessel permeability after intracarotid bradykinin infusion is mediated by nitric oxide, Cancer Research, 56:4027-4031 [1996]; Faraci, F. M. et al., Role of soluble guanylate cyclase in dilator responses of the cerebral microcirculation, Brain Res. 821(2): 368-73 [1999]). Further, the pretreatment of glioma-bearing rats with the NOS inhibitor, L-NAME, significantly reduces bradykinin-induced permeability. (Moncada, S. et al., Endogenous nitric oxide: physiology, pathology and clinical relevance, Eur. J. Clin. Invest. 21(4)361-74 [1991]; Sugita, M. et al., Nitric oxide and cyclic GMP attenuate sensitivity of the tumor barrier to bradykinin, Neurological Research 20: 559-563 [1998]).

In turn, one class of enzymes that is activated by cGMP (and cAMP) is cGMP-dependent protein kinases (PKG or cGK), which through enzymatic ATP-dependent phosphorylation, directly or indirectly activate calcium-dependent potassium channels (Robertson, B. E. et al., cGMP-dependent protein kinase activates Ca-activated K channels in cerebral artery smooth muscle cells, Am. J. Physiol. 265[Cell Physiol. 34):C299-C303 [1993]; Fukao, M. et al., Cyclic GMP-dependent protein kinase activates cloned BK_{Ca} channels expressed in mammalian cells by direct phosphorylation at serine 1072, J. Biol. Chem. 274(16): 10927-35 [1999]; Becker, E. M. et al., The vasodilator-stimulated phosphoprotein (VASP): target of YC-1 and nitric oxide effects in human and rat platelets, J. Cardiovasc. Pharmacol.35(3): 390-97 [2000]). There is also evidence that nitric oxide can activate K_{Ca} by both cGMP-dependent and cGMP-independent mechanisms. (Chen, C. H. et al., Nitric oxide activates Ca²⁺-activated K⁺ channels in cultured bovine adrenal chromaffin cells, Neurosci. Lett. 248(2): 127-29 [1998]; Vaali, K. et al., Relaxing effects of NO donors on guinea pig trachea in vitro are mediated by calcium-sensitive potassium channels, J. Pharmacol. Exp. Ther. 286(1): 110-14 [1998]; Sobey, C. G. and Faraci, F. M., Inhibitory effect of 4-aminopyridine on responses of the basilar artery to nitric oxide, Br. J. Pharmacol. 126(6): 1437-43 [1999]; Kurtz, A. et al., Mode of nitric oxide action on the renal vasculature, Acta Physiol. Scand. 168(1): 41-45 [2000]).

Treatments directed to the use of potassium channel activators or agonists have been taught for disorders including hypertension, cardiac and cerebral ischemia, nicotine addiction, bronchial constriction, and neurodegenerative diseases, but not particularly for the treatment of malignant tumors. (Erhardt et al., Potassium channel activators/openers, U.S. Pat. No. 5,416,097; Schohe-Loop et al., 4,4′-bridged bis-2,4-diaminoquinazolines, U.S. Pat. No. 5,760,230; Sit et al., 4-aryl-3-hydroxyquinolin-2-one derivatives as ion channel modulators, U.S. Pat. No. 5,922,735; Garcia et al., Biologically active compounds, U.S. Pat. No. 5,399,587; Cherksey, Potassium channel activating compounds and methods of use thereof, U.S. Pat. No. 5,234,947).

Bradykinin is thought to increase [Ca²⁺]ᵢ and thus may activate K_{Ca} channels. While some other known activators of K_{Ca} do not act as vasodilators, for example, 1,3-dihydro-1-[2-hydroxy-5-(trifluoromethyl)phenyl]-5-(trifluoromethyl)-2H-benzimidazol-2-one (NS-1619; M. Holland et al., Effects of the BK_{Ca} channel activator, NS1619, on rat cerebral artery smooth muscle, Br. J. Pharmacol., 117(1): 119-29 [1996]), evidence is accumulating that K_{Ca} may play an important role in vasodilatation mediated by vasodilators, such as bradykinin, nitric oxide donors, cyclic guanosine monophosphate (cGMP), and guanylyl cyclase activators. (Berg T., Koteng O., Signaling pathways in bradykinin-and nitric oxide-induced hypotension in the normotensive rat; role of K⁺-channels, Br. J. Pharmacol.; 121(6): 1113-20 [1997]; Bolotina, V. M. et al., Nitric oxide directly activates calcium-dependent potassium channels in vascular smooth muscle, Nature 368(6474): 850-3 [1994]; Robertson, B. E., et al., cGMP-dependent protein kinase activates Ca-activated K channels in cerebral artery smooth muscle cells, Am. J. Physiol. 265(1 Pt 1):C299-303 [1993]; Sobey, C. G. et al., Mechanisms of bradykinin-induced cerebral vasodilatation in rats. Evidence that reactive oxygen species activate K⁺ channels, Stroke 28(11): 2290-4; discussion 2295 [1997]; C. G. Sobey and F. M. Faraci, Effect of nitric oxide and potassium channel agonists and inhibitors on basilar artery diameter, Am. J. Physiol. 272(1 Pt 2):H256-62 [1997]; Hardy, P. et al., A major role for prostacyclin in nitric oxide-induced ocular vasorelaxation in the piglet, Circ. Res. 83(7): 721-29 [1998]; Bychkov R. et al., Calcium-activated potassium channels and nitrate-induced vasodilation in human coronary arteries, J. Pharmacol. Exp. Ther. 285(1): 293-98 [1998]; Armstead, W. M., Contribution of K_{Ca} Channel activation to hypoxic cerebrovasodilation does not involve NO, Brain Res. 799(1): 44-48 [1998]).

Bradykinins action as a powerful vasodilator is disadvantageous when using bradykinin to open the blood-brain barrier to therapeutic anticancer agents. Bradykinin or its analogs may adversely lower blood pressure, reduce cerebral blood flow, or contribute to brain edema in some patients. (E.g., A. M. Butt, Effect of inflammatory agents on electrical resistance across the blood-brain barrier in pial microvessels of anesthetized rats, Brain Res. 696(1-2): 145-50 [1995]). In addition, bradykinin constricts smooth muscle and stimulates pain receptors.

Consequently, there is still a definite need to maximize the effectiveness of a wide variety of therapeutic agents through enhanced selective transvascular delivery to malignant tumors, including those of the central nervous system, and/or to other abnormal brain regions. These and other benefits the present invention provides as described herein.

### SUMMARY OF THE INVENTION

The present invention relates to a method of delivering a medicant to an abnormal brain region in a mammalian subject, including a human. The method includes administering to the subject a potassium channel activator. Potassium channel activators include direct agonists (other than bradykinin or bradykinin analogs), such as NS-1619 or minoxidil. Potassium channel activators also include compounds that indirectly activate potassium channels, for example nitric oxide, nitric oxide donors, and other activators of guanylyl cyclase. Activators of cyclic GMP-dependent protein kinase, which activates calcium-dependent potassium channels, are also included. The potassium channel activator is administered to the subject under conditions and in an amount sufficient to increase the permeability to the medicant of a capillary or arteriole delivering blood to cells of the abnormal brain region in the subject. Simultaneously or substantially simultaneously with the potassium channel activator, the medicant is administered, so that the medicant is delivered selectively to the cells of the abnormal region compared to normal brain regions, due to the increased permeability of capillaries and arterioles supplying the abnormal brain region. The method is particularly valuable in the treatment of physical or biochemical brain injuries caused by trauma, infection, stroke, ischemia, and, particularly, malignant brain tumors, for which survival rates are notoriously poor.

The present invention also relates to a method of delivering a medicant to a malignant tumor in the brain or anywhere in the body of a mammalian subject. The method involves administering to the subject a potassium channel activator, other than bradykinin or a bradykinin analog, under conditions and in an amount sufficient to increase the permeability to the medicant of a capillary or arteriole delivering blood to cells of the malignant tumor in the subject. Simultaneously or substantially simultaneously with the potassium channel activator the medicant is administered to the subject, and it is delivered selectively to the malignant cells compared to non-malignant cells by virtue of the potassium channel activator. The inventive method is useful in treating any kind of malignant tumor by increasing the selectivity of drug delivery to neoplastic tissue, thereby minimizing damage to non-malignant tissue from medicants, including cytotoxic chemotherapeutic agents, and focusing the therapeutic or diagnostic action of the agents. Thus, this invention, also directed to a method of treating a malignant tumor in a human subject, offers enhanced prospects of survival to cancer patients, with fewer harmful side effects. The selectivity of the methods is based on the role of calcium-and ATP-dependent potassium transporters (channels) in mediating the permeability of microvasculature to various drugs, macromolecules, and viral particles, combined with the greater number of calcium- and ATP-dependent potassium channels present in abnormal brain vasculature or tumor neomicrovasculature compared to normal microvasculature.

The present invention also relates to a pharmaceutical composition that comprises a combination of a potassium channel activator, other than bradykinin or a bradykinin analog, formulated in a pharmaceutically acceptable solution together with a medicant for delivery by intravascular infusion or bolus injection into a mammal, such as a human. The pharmaceutical composition is useful in practicing the inventive methods.

The invention also relates to a kit for enhancing the delivery of a medicant to an abnormal brain region and/or to a malignant tumor.

These and other advantages and features of the present invention will be described more fully in a detailed description of the preferred embodiments which follows. In further describing the invention, the disclosures of related application U.S. Ser. No. 09/491,500 are incorporated herein by reference.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 shows the response to the soluble guanylyl cyclase activator YC-1 (2.66 μg min⁻¹kg⁻¹; n=6) in the unidirectional transfer constant Kᵢ for [¹⁴C]α-aminoisobutyric acid in malignant RG2 glioma tissue in Wistar rats, compared to DMSO+saline control (n=6), YC-1+iberiotoxin (IBTX; 0.2 μg IBTX min⁻¹kg⁻¹; n=3), or YC-1+a selective inhibitor of soluble guanylyl cyclase, 1H-[1,2,4]oxadiazolo[4,3-a]quinozalin-1-one (ODQ) (2.66 μg min⁻¹kg⁻¹; n=4) treatments for 15 minutes each. Kᵢ (μL/g/min) in the tumor core (gray bars), tumor-adjacent tissue taken about 3 mm from the tumor core (white bars), and normal contralateral brain tissue (black bars) is shown.

FIG. 2 shows the enhancing effect of the nitric oxide donor diethylamine-NONOate (DEA/NO) on blood-tumor barrier permeability to Evans blue stain in malignant RG2 glioma tissue in Wistar rats. FIG. 2A shows a brain section from a rat treated with phosphate buffered saline (PBS only control). FIG. 2B shows a brain section from a DEA/NO-treated rat.

FIG. 3 shows the response to DEA/NO (2.66 μg min⁻¹kg⁻¹; n=3) in the unidirectional transfer constant Kᵢ for [¹⁴C]α-aminoisobutyric acid in malignant RG2 glioma tissue in Wistar rats, compared to PBS control, DEA/NO+iberiotoxin (IBTX; 0.2 μg IBTX min⁻¹kg⁻¹; n=3), or PAPA/NO (2.66 μg min⁻¹kg⁻¹; n=3) treatments for 15 minutes each. K, (μL/g/min.) in tumor centers (black bars), tumor-adjacent tissue taken about 3 mm from the tumor core (white bars), and normal contralateral brain tissue (cross-hatched bars) is shown.

FIG. 4 shows transendothelial vesicular transport in a RG2 tumor capillary. RG2 tumor-bearing brain sections from Wistar rats were prepared after a 15-minute intracarotid infusion with: PBS (0.8 mL)(FIG. 4A; 54,000× magnification); bradykinin (10 μg min⁻¹kg⁻¹)(FIG. 4B; 87,000× magnification); or NS-1619 (5.3 μg min⁻¹‘kg-’; FIG. 4C; 87,000× magnification). TEM analysis of the tumor center section shows that K_{Ca} activators bradykinin and NS-1619 increase pinocytotic transport vesicles in the endothelial cytoplasm.

FIG. 5 shows that a potassium channel activator (bradykinin) increases transport of horse radish peroxidase (HRP) from the tumor capillary lumen through endothelial pinocytotic vesicles. FIG. 5A (8,700× magnification) and FIG. 5B (21,000× magnification) show vascular and tumor tissue from a rat in the PBS control group. FIG. 5C (8,700× magnification) and FIG. 5D (21,000× magnification) show vascular and tumor tissue from a rat treated with the potassium channel activator bradykinin.

FIG. 6 illustrates that a potassium channel activator (bradykinin) induces vesicular transport of HRP in RG2 tumor cells. FIG. 6A (8,700× magnification) shows tumor tissue from a rat in the PBS control; FIG. 6B (10,800× magnification) and FIG. 6C (21,000× magnification) show tumor tissue from a bradykinin-treated rat.

FIG. 7 shows an increase in the unidirectional transport constant caused by the K_{ATP} activator minoxidil sulfate and the K_{Ca} activator bradykinin.

FIG. 8 shows that bradykinin-induced increase in Kᵢ was not attenuated by glibenclamide, while IBTX significantly decreased the effect caused by bradykinin.

FIG. 9 shows that the minoxidil sulfate-induced increase in Kᵢ was attenuated by glibenclamide, while IBTX failed to block the effect.

FIG. 10 illustrates increased microvascular permeability in an ischemic brain region as a result of treatment with a potassium channel activator. Kᵢ is shown for bradykinin-treated (black bars) and control animals (gray bars) in paired histograms; tissues tested were (left to right): caudate putamen; cortex; caudate putamen contralateral side; and cortex contralateral side. FIG. 10A shows that blood-brain barrier permeability after one hour of ischemia was not affected by bradykinin treatment compared to the vehicle-treated group (MCA occlusion for 1 hour, followed by 1 hour of reperfusion; Inset: autoradiograph). FIG. 10B shows that bradykinin significantly increased permeability in ischemic infarct penumbra after 2 hours of MCA occlusion, followed by 1 hour of reperfusion (Inset: autoradiograph).

FIG. 11 shows that potassium activators enhance permeability of microvasculature in ischemic brain tissue. No change in blood-brain barrier permeability at the infarct brain tissue (significantly less uptake of Evans blue) was observed in a rat subjected to temporary ischemia (2 hours MCA occlusion and 1 hour of reperfusion; top and bottom left panels). Considerable enhancement of Evans blue uptake was seen in ischemic brain tissue in bradykinin-treated (10 μg min⁻¹kg⁻¹ for 15 min; top middle panel) or NS-1619-treated (1.5 μg min⁻¹kg⁻¹ for 15 min; lower middle panel) rats compared to PBS vehicle-treated (top and bottom left panels). However, co-infusion with IBTX (0.2 μg min⁻¹kg⁻¹ for 15 min) attenuated the increased uptake of Evans blue induced by bradykinin (top right panel) or NS-1619 (bottom right panel).

### DETAILED DESCRIPTION OF THE PREFERRED EMBODIMENTS

The inventive methods are useful for selectively delivering a medicant to abnormal brain regions and/or malignant tumors in mammalian subjects. The methods involve administering to the mammalian subject a potassium channel activator, other than bradykinin or a bradykinin analog, under conditions and in an amount sufficient to increase the permeability to the medicant of a capillary or arteriole delivering blood to cells of the abnormal brain region and/or to malignant cells of a malignant tumor present in the subject. The increase in permeability ranges from at least 2-to 6-fold, compared to controls without the administration of a potassium channel activator. The relative increase in permeability tends to be greater for large molecular weight medicants (e.g., about 10,000 to 250,000 Daltons) than for smaller molecular weight substances (e.g., about 50-200 Daltons).

The abnormal brain regions include regions of brain tissue physiologically directly affected by a physical or biochemical injury, for example, Alzheimers disease, Parkinsonism, trauma, infection, stroke, brain ischemia, or regions of neoplastic growth within the brain, such as benign or malignant brain tumor tissues.

The present invention is also useful for selectively delivering a medicant to a malignant tumor in the brain or to a tumor elsewhere in the body of a mammalian subject. The inventive technology is useful in the treatment of all kinds of solid malignant tumors, including gliomas, glioblastomas, oligodendrogliomas, astrocytomas, ependymomas, primitive neuroectodermal tumors, atypical meningiomas, malignant meningiomas, neuroblastomas, sarcomas, melanomas, lymphomas, or carcinomas. The tumor to be treated can be contained in the skull, brain, spine, thorax, lung, peritoneum, prostate, ovary, uterus, breast, stomach, liver, bowel, colon, rectum, bone, lymphatic system, skin, or in any other organ or tissue of the subject.

The inventive methods are useful in treating any mammal, including a human, non-human primate, canine, feline, bovine, porcine or ovine mammal, as well as in a small mammal such as a mouse, rat, gerbil, hamster, or rabbit.

The potassium channel activator is an activator of either a calcium-activated potassium channel (K_{Ca}) of any conductance level, whether of large, intermediate, or small conductance, or of an ATP-sensitive potassium channel (K_{ATP}). Included are direct agonists of K_{Ca}, such as 1,3-dihydro-1-[2-hydroxy-5-(trifluoromethyl)phenyl]-5-(trifluoromethyl)-2H-benzimidazol-2-one (NS-1619) or 1-ethyl-2-benzimidazolinone (1-EBIO).

Also included among useful potassium channel activators are compounds that indirectly activate potassium channels, for example activators of soluble guanylyl (i.e., guanylate) cyclase, such as nitric oxide, nitric oxide donors, metalloporphyrins (e.g., zinc or tin protoporphyrin IX), YC-1 (a benzyl indazole derivative), or guanylyl cyclase activating proteins (GCAPs). (See, e.g., Koesling, D., Modulators of soluble guanylyl cyclase, Naunyn-Schmiedebergs Arch. Pharmacol.358:123-126 [1998]).

Examples of useful potassium channel activators that are K_{ATP} agonists include minoxidil (2,4-diamino-6-piperidino pyramidine-3-oxide; insoluble in water, soluble in ethanol 29 mg/mL), pinacidil ((+/−)-N-cyano-N′-4-pyridinyl-N″-(1,2,2-trimethyl propyl)-guanidine; insoluble in water, soluble in ethanol 14 mg/mL]), (+)-cromakalim, (−)-cromakalim or levcromakalim, (+/−)-cromakalim, or diazoxide.

A preferred potassium channel activator is nitric oxide gas, which is fully permeable across biological membranes. Inhalable nitric oxide gas can be administered to the subject by mask in a controlled gas mixture as is known in the art. (E.g., Kieler-Jensen, N. et al., Inhaled nitric oxide in the evaluation of heart transplant candidates with elevated pulmonary vascular resistance, J Heart Lung Transplant. 13(3): 366-75 [1994]; Rajek, A. et al., Inhaled nitric oxide reduces pulmonary vascular resistance more than prostaglandin E(1) during heart transplantation, Anesth Analg. 90(3): 523-30 [2000]; Solina, A. et al., A comparison of inhaled nitric oxide and milrinone for the treatment of pulmonary hypertension in adult cardiac surgery patients, J Cardiothorac Vasc. Anesth. 14(1): 12-17 [2000]; Fullerton, D. A. et al., Effective control of pulmonary vascular resistance with inhaled nitric oxide after cardiac operation, J Thorac Cardiovasc Surg 111(4): 753-62, discussion 762-3 [1996]). The concentration in the gas mixture of nitric oxide (NO) is preferably about 1 to 100 ppm NO, more preferably about 4 to 80 ppm NO, and most preferably about 20 to 40 ppm NO. The gas mixture also contains appropriate concentrations of oxygen and nitrogen and/or other inert gases, such as carbon dioxide, helium or argon. Optionally, gaseous anesthetics, such as nitrous oxide (N₂O), xenon, and halogenated volatile anesthetics (HVAs), e.g., halothane, sevoflurane, and isoflurane, are also included in the gas mixture when general anesthesia is indicated. General anesthesia is indicated, for example, when administration of the potassium channel activator (and/or the medicant or chemotherapeutic agent) is by intracarotid infusion; general anesthesia is typically not required using intravenous or other delivery routes. The skilled practitioner is aware of evidence that HVAs can inhibit soluble guanylyl cyclase activity. (See, Masaki, E., Halogenated volatile anesthetics inhibit carbon monoxide-stimulated soluble guanylyl cyclase activity in rat brain, Acta Anaesthesiol. Scand. 44(3): 321-25 [20001; Masaki E. and Kondo I, Methylene blue, a soluble guanylyl cyclase inhibitor, reduces the sevoflurane minimum alveolar anesthetic concentration and decreases the brain cyclicguanosine monophosphate content in rats, Anesth. Analg. 89(2): 484-89 [1999]). Consequently, an HVA is not the preferred choice of inhalable anesthesia for use with a guanylyl cyclase activator in accordance with the method.

Nitric oxide donors are compounds that produce NO-related physiological activity when applied to biological systems. Thus, NO-donors can mimic an endogenous NO-related response or substitute for an endogenous NO deficiency. The skilled artisan is aware that in biological systems there are at least three redox states of NO that can be released by various NO donors (NO⁺, NO⁰, or NO⁻), all of which are encompassed by the terms “nitric oxide” or “NO” for purposes of the present invention. The redox state of NO makes a substantial difference to the NO donors reactivity towards other biomolecules, the profile of by-products, and the bioresponse (Feelisch, M., The use of nitric oxide donors in pharmacological studies, Naunyn-Schmiedebergs Arch. Pharmacol.358:113-22 [1998]). Some classes of NO donors require enzymatic catalysis, while others produce NO non-enzymatically; some NO donors require reduction, for example by thiols, and some oxidation, in order to release NO.

Preferred examples of nitric oxide donors include organic nitrate compounds, which are nitric acid esters of mono- and polyhydric alcohols. Typically, these have low water solubility, and stock solutions are prepared in ethanol or dimethyl sulfoxide (DMSO). Examples are glyceryl trinitrate (GTN) or nitroglycerin (NTG), pentaerythrityl tetranitrate (PETN), isosorbide dinitrate (ISDN), and isosorbide 5-mononitrate (IS-5-N). Administration of organic nitrates can be done intravenously, intraperitoneally, intramuscularly, transdermally, or in the case of PETN, ISDN, NTG, and IS-5-N, orally.

Other preferred examples are S-nitrosothiol compounds, including S-nitroso-N-acetyl-D,L-penicillamine (SNAP), S-nitrosoglutathione (SNOG), S-nitrosoalbumin, S-nitrosocysteine. S-nitrosothiol compounds are particularly light-sensitive, but stock solutions kept on ice and in the dark are stable for several hours, and chelators such as EDTA can be added to stock solutions to enhance stability. Administration is preferably by an intravenous or intra-arterial delivery route.

Other preferred examples of nitric oxide donors include sydnonimine compounds, such as molsidomine (N-ethoxycarbonyl-3-morpholino-sydnonimine), linsidomine (SIN-1; 3-morpholino-sydnonimine or 3-morpholinylsydnoneimine or 5-amino-3morpholinyl-1,2,3-oxadiazolium, e.g., chloride salt), and pirsidomine (CAS 936). Stock solutions are typically prepared in DMSO or DMF, and are stable at 4° C. to room temperature, if protected from light. Linsidomine is highly water soluble and stable in acidic solution in deoxygenated distilled water, adjusted to about pH 5, for an entire day. At physiological pH, SIN-1 undergoes rapid non-enzymatic hydrolysis to the open ring form SIN-1A, also a preferred nitric oxide donor, which is stable at pH 7.4 in the dark. Administration is preferably by an intravenous or intra-arterial delivery route.

Also useful as nitric oxide donors are iron nitrosyl compounds, such as sodium nitroprusside (SNP; sodium pentacyanonitrosyl ferrate(II)). Aqueous stock solutions are preferably made freshly in deoxygenated water before use and kept in the dark; stability of stock solutions is enhanced at pH 3-5. Inclusion in the delivery buffer of a physiologically compatible thiol, such as glutathione, can enhance release of NO. SNP is administered by intravenous infusion, and the skilled practitioner is aware that long-term use is precluded by the release of five equivalents of toxic CN-per mole SNP as NO is released.

A most preferred nitric oxide donor is chosen from among the so-called NONOate compounds. The NONOates are adducts of NO with nucleophilic residues (X⁻), such as an amine or sulfite group, in which an NO dimer is bound to the nucleophilic residue via a nitrogen atom to form a functional group of the structure X[—N(O)NO]⁻. The NONOates typically release NO at predictable rates largely unaffected by biological reactants, and NO release is thought to be by acid-catalyzed dissociation with the regeneration of X⁻ and NO. This property is particularly useful in accordance with the inventive methods of selectively delivering a medicant, because abnormal brain regions and malignant tumors can typically be relatively hypoxic and possess a relatively low ambient pH (e.g., pH 6.5-7.0), which concentrates release of NO selectively in the microvasculature of the abnormal brain region or malignant tumor.

NONOates include most preferably diethylamine-NONOate (DEA/NO; N-Ethylethanamine: 1,1-Diethyl-2-hydroxy-2-nitrosohydrazine (1:1) or 1-[N,N-diethylamino]diazen-1-ium-1,2-diolate). Other preferred NONOates include diethylene triamine-NONOate(DETA/NO; 2,2′-Hydroxynitrosohydrazino]bis-ethanamine), spermine-NONOate (SPER/NO; N-(4-[-1-(3-Aminopropyl)-2-hydroxy-2-nitrosohydrazino]butyl)-1,3-propanediamine), propylamino-propylamine-NONOate (PAPA/NO; 3-(2-Hydroxy-2-nitroso-1-propylhydrazino)-1-propanamine or (Z)-1-[N-(3-aminopropyl)-N-(n-propyl)amino]diazen-1-ium-1,2-diolate), MAHMA-NONOate (MAHMA/NO; 6-(2-Hydroxy-1-methyl-2-nitrosohydrazino)-N-methyl-1-hexanamine), dipropylenetriamine-NONOate (DPTA/NO; 3,3′-(Hydroxynitrosohydrazino)bis-1-propanamine), PIPERAZI/NO, proli-NONOate (PROLI/NO; 1-([2-carboxylato]pyrrolidin-1-yl)diazen-1-ium-1,2-diolate-methanol, e.g., disodium salt), SULFO-NONOate (SULFO/NO; hydroxydiazenesulfonic acid 1-oxide, e.g., diammonium salt), the sulfite NONOate (SULFI/NO), and Angelis salt (OXI/NO).

Almost all NONOate compounds are highly soluble in water, and aqueous stock solutions are prepared in cold deoxygenated 1 to 10 mM NaOH (preferably about pH 12) just prior to use. Alkaline stock solutions are stable for several hours if kept on ice in the dark. The characteristeic UV absorbance of NONOates can be used for spectrophotometric quantification of NONOate in aqueous solutions. NONOates are preferably administered intravenously or intra-arterially.

Nitric oxide donors have different potencies (Ferraro, R. et al., Comparative effects of several nitric oxide donors on intracellular cyclic GMP levels in bovine chromaffin cells: correlation with nitric oxide production, Br. J. Pharmacol. 127(3): 779-87 [1999]). For example, DEA/NO is among the most potent nitric oxide donors, with a half-life of about 2 to 4 minutes; less potent are PAPA/NO (t_{1/2} about 15 minutes),SPER/NO (t_{1/2} about 34-40 minutes); even less potent are DETA/NO (t_{1/2} about 20 hours) and SNAP (t_{1/2} about 33 to 41 hours, although this can be shortened in the presence of a physiological reductant such as glutathione). SNP is also a potent NO donor. (See, Ferrero et al. [1999); Salom, J. B. et al., Relaxant effects of sodium nitroprusside and NONOates in rabbit basilary artery, Pharmacol. 57(2): 79-87 [1998]; Salom, J. B. et al., Comparative relaxant effects of the NO donors sodium nitroprusside, DEA/NO and SPER/NO in rabbit carotid arteries, Gen. Pharmacol. 32(1): 75-79 [1999]; Salom, J. B. et al., Relaxant effects of sodium nitroprusside and NONOates in goat middle cerebral artery: delayed impairment by global ischemia-reperfusion, Nitric Oxide 3(1): 85-93 (1999]; Kimura, M. et al., Responses of human basilar and other isolated arteries to novel nitric oxide donors, J. Cardiovac. Pharmacol. 32(5): 695-701 [1998]). Consequently, effective concentrations or doses of NONOates or other NO donors will vary over the preferred dose ranges for potassium channel activators described herein.

Stock solutions of NO donors are preferably made up freshly before use (at the appropriate pH for each particular NO donor), chilled on ice, and protected from light (e.g., by the use of darkened glass vials wrapped in aluminum foil), although organic nitrates can be stored for months to years if the vial is properly sealed. Preferably, immediately before administration to the subject, final dilutions are prepared in pharmaceutically acceptable buffer and the final pH of the NO donor-containing buffer is checked for physiological suitability, especially when strongly acidic (e.g., hydrochloride salts) or alkaline (e.g., NONOates) stock solutions are used.

The product of NO exposure time and NO concentration largely determines the quality and magnitude of the biological response to exogenously supplied NO. Short-lived NO donors, such as DEA/NO, are most preferably administered by continuous infusion rather than by bolus to avoid delivering only a short burst of NO.

Also included among potassium channel activators are activators of any endogenous species of cyclic GMP-dependent protein kinase (PKG or cGK), that activates a potassium channel directly (e.g., by directly phosphorylating K_{Ca}) or indirectly (e.g., by phosphorylating another regulatory protein that directly modulates K_{Ca} activity). Included are activators of cGK I, cGK II, or other isoforms of cGMP-dependent protein kinase. (e.g., Smolenski, A. et al. [1998]). Useful examples of PKG activators include, but are not limited to, octobromo-cyclic GMP (8Br-cGMP) and dibutyryl cyclic GMP.

Included among useful potassium channel activators are pharmaceutically acceptable molecular conjugates or salt forms that still have activity as potassium channel activators as defined herein. An example is minoxidil sulfate, but other pharmaceutically acceptable salts comprise anions other than sulfate, such as chloride, carbonate, bicarbonate, nitrate, or the like. Other embodiments of pharmaceutically acceptable salts contain cations, such as sodium, potassium, magnesium, calcium, ammonium, or the like. Other embodiments of useful potassium channel activators are hydrochloride salts.

However, the potassium channel activator employed in the inventive methods is one other than the vasodilator bradykinin (Arg-Pro-Pro-Gly-Phe-Ser-Pro-Phe-Arg), or a polypeptide bradykinin analog, such as receptor mediated permeabilizer (RMP)-7 or A7 (e.g., Kozarich et al., U.S. Pat. No. 5,268,164 and PCT Application No. WO 92/18529). Other analogs of bradykinin include related peptide structures which exhibit the same properties as bradykinin but have modified amino acids or peptide extensions on either terminal end of the peptide. Examples of bradykinin analogs include [phe.sup.8 (CH.sub.2 NH) Arg.sup.9]-bradykinin, Nacetyl [phe.sup.8 (CH.sub.2—NH—Arg.sup.9] bradykinin and desArg9-bradykinin.

In accordance with the inventive methods, the potassium channel activator is administered by intravenous or intra-arterial injection or infusion. For treating an abnormal brain region, such as an intracranial tumor, the potassium channel activator is preferably administered by intracarotid infusion. Except where otherwise noted, the amount of potassium channel activator to be administered to the mammalian subject ranges from 0.075 to 1500 micrograms per kilogram body mass. For humans the range of 0.075 to 150 micrograms per kilogram body mass is preferred. As the skilled practitioner is aware, the physiological responses of individual patients to treatment with particular potassium channel activators will vary. For example, generally an effective amount of YC-1 for humans is about 15 to about 45 micrograms per kg body mass, and for nitric oxide donors generally about 15 to about 45 micrograms per kg body mass. However, the optimal amount for each individual for any particular potassium channel activator can be determined by routine means involving close physiological monitoring over the delivery period.

The dose can be administered in a bolus injection, but is preferably administered by infusion over a period of one to thirty minutes, and most preferably during a period of one to fifteen minutes. For example, in rats, a dose rate of about 0.75 to about 100 μg kg⁻¹min⁻¹ is most suitable. At dose rates above about 100 μg kg⁻¹min⁻¹ a concomitant fall in blood pressure has sometimes been observed. In humans, effective dose rates are about 0.075 to about 15 μg kg⁻¹min⁻¹, with cautious monitoring of blood pressure being advised. The practitioner skilled in the art is also cautious in regulating the total infusion volume, rate of liquid infusion, and electrolyte balance to avoid adverse physiological effects related to these.

Some potassium channel activators, such as NS-1619, minoxidil, minoxidil sulfate, pinacidil, or diazoxide are not easily dissolved in water; in preparing these agents for administration, a suitable and pharmaceutically acceptable solvent, such as ethanol, can be used to dissolve the potassium channel activator prior to further dilution with an infusion buffer. The skilled practitioner is cautious in regulating the final concentration of solvent in the infusion solution to avoid solvent-related toxicity. For example, a final ethanol concentration in an infusion solution up to 5-10% (v/v) is tolerated by most mammalian subjects with negligible toxicity.

While the inventive method does not depend on any particular mechanism by which increased microvascular permeability to the medicant is achieved, it is thought that administration of the potassium channel activator increases potassium flux through potassium channels in endothelial cell membranes of the capillaries and arterioles delivering blood to abnormal brain regions and/or malignant tumors. This results in a loosening of tight junctions in the microvascular epithelium and/or increased pinocytotic activity, enhancing the uptake of medicants from the blood vessels. In practicing the inventive methods, it is not necessary to measure potassium channel activity (i.e., potassium cation flux therethrough). But the skilled artisan is aware that potassium flux can be measured by any suitable method, for example, by measuring cellular uptake of ⁴²K⁺ or ²⁰¹Tl⁺ or channel conductance using patch-clamp or microelectrode devices. (e.g., T. Brismar et al., Thallium-201 uptake relates to membrane potential and potassium permeability in human glioma cells, Brain Res. 500(1-2): 30-36 [1989]; T. Brismar et al., Mechanism of high K⁺ and Tl⁺ uptake in cultured human glioma cells, Cell Mol. Neurobiol. 15(3): 351-60 [1995]; S. Cai et al., Single-channel characterization of the pharmacological properties of the K(Ca2⁺) channel of intermediate conductance in bovine aortic endothelial cells, J. Membr. Biol. 163(2): 147-58 [1998]).

The medicant is administered simultaneously or substantially simultaneously with the potassium channel activator, and the medicant is delivered by the blood stream selectively to the abnormal brain region and/or to the malignant cells compared to normal brain tissue or non-malignant cells. “Simultaneously” means that the medicant is administered contemporaneously or concurrently with the potassium channel activator. “Substantially simultaneously” means that the medicant is administered within about one hour after the potassium channel activator is last administered, preferably within about 30 minutes after, and most preferably, is administered simultaneously with the potassium channel activator. Alternatively, “substantially simultaneously” means that the medicant is administered within about 30 minutes before, and preferably within about 15 minutes before the potassium channel activator is first administered.

The methods of delivering a medicant to an abnormal brain region and/or to a malignant tumor in a mammalian subject are effective in selectively delivering any medicant across the microvascular of an abnormal brain region and/or malignant tumor. The medicant is a drug, i.e., a chemotherapeutic agent. Example of chemotherapeutic agents including therapeutic cytotoxic agents (e.g., cisplatin, carboplatin, methotrexate, 5-fluorouracil, amphotericin), naked DNA expression vectors, therapeutic proteins, therapeutic oligonucleotides or nucleotide analogs, interferons, cytokines, or cytokine agonists or antagonists, adrenergic agents, anticonvulsants, anti-trauma agents, or any neuropharmaceutical agent used to treat or prevent an injury or disorder of the brain. Chemotherapeutic agents also include ischemia-protective drugs such as N-methyl-D-aspartate (NMDA) receptor antagonists; antimicrobial agents, such as antibiotics; immunotoxins, immunosuppressants, boron compounds, monoclonal antibodies and specific antigen-binding antibody fragments (e.g., Fab, Fab′, F(ab′)₂, or F(v) fragments), and cytokines, such as interferons, interleukins (e.g., interleukin [IL]-2), tumor necrosis factor (TNF)-α, or transforming growth factors (e.g., TGF-β).

The medicant also includes anticancer chemotherapeutic agents. Typically, anticancer chemotherapeutic agents are cytotoxic agents, such as 5-fluorouracil, cisplatin, carboplatin, methotrexate, daunorubicin, doxorubicin, vincristine, vinblastine, or a cytotoxic alkylating agent, such as, but not limited to, busulfan (1,4-butanediol dimethanesulphonate; Myleran, Glaxo Wellcome), chlorambucil, cyclophosphamide, melphalan, or ethyl ethanesulfonic acid.

The anticancer chemotherapeutic agents are particularly useful in practicing the method of selectively delivering a medicant to a malignant tumor, in the brain or in any other tissue of the body, and in the method of treating a malignant tumor in a human subject.

Medicants also include any therapeutic viral particle, for example an adenovirus-derived or herpes simplex virus (HSV)-derived viral vector for delivering genetic material to a cellular target in vivo. Medicants also include diagnostic agents, such as imaging or contrast agents, for example, radioactively labeled substances (e.g., [⁹⁹Tc]-glucoheptonate), gallium-labeled imaging agents (e.g., gallium-EDTA), ferrous magnetic, fluorescent, luminescent, or iodinated contrast agents. Where suitable, any of the afore-mentioned medicants having anticancer activity can also be used in practicing the method of selectively delivering a medicant to a malignant tumor or the method of treating a malignant tumor in a human subject.

Thus, the medicant can be a molecular substance having a molecular weight between about 50 Daltons and about 250 kD. Or it can be a particle, such as a viral particle, having a diameter between about 50 to 250 nanometers.

This is by no means intended to be an exhaustive list of the kinds of medicants that can be employed in practicing the inventive methods. The medicant can be, but is preferably not, an agent that is highly lipid soluble and thus inherently able to penetrate cell membranes, for example nitrosourea.

The amount of medicant that is employed is within a conventional dose range for each medicant, however by practicing the inventive method, the increased transvascular permeability afforded can provide a greater selective therapeutic effect per dose or permit a lower effective dose to be used, if desired, for example to lessen systemic toxic effects from anti-cancer medication in a particular subject.

The medicant is administered by any appropriate method that can deliver it to the blood stream. Typically, this is by intravenous, intramuscular, or intra-arterial (including intracarotid) injection or infusion. However, for some applications other acceptable delivery routes can be used as long as the dose of medicant enters the blood stream substantially simultaneously with the potassium channel activator. Examples include ingestion (e.g., of a powder, suspension, solution, emulsion, tablet, capsule or caplet); subcutaneous injection; stereotactic injection; or transdermal or transmucosal delivery by adhesive patch, suppository or gel for delivery through the skin, mucosa or epithelium of the mouth including the sublingual epithelium, the rectum, or the vaginal epithelium.

Alternatively, the medicant is administered together with the potassium channel activator in a pharmaceutical composition of the present invention. The inventive pharmaceutical composition comprises a combination of a potassium channel activator, other than bradykinin or a bradykinin analog, as described above, formulated in a pharmaceutically acceptable solution together with a medicant, as described above, for delivery by intravascular infusion or bolus injection into a mammal, such as a human. The solution is thus suitably balanced, osmotically (e.g., about 0.15 M saline) and with respect to pH, typically between pH 7.2 and 7.5; preferably the solution further comprises a buffer, such as a phosphate buffer (e.g., in a phosphate buffered saline solution). The solution is formulated to deliver a dose rate of about 0.075 to 1500 micrograms of potassium channel activator per kilogram body mass in a pharmaceutically acceptable fluid volume over a maximum of about thirty minutes. For human subjects, the solution is preferably formulated to deliver a dose rate of about 0.075 to 150 micrograms of potassium channel activator per kilogram body mass in a pharmaceutically acceptable fluid volume over a period of up to about thirty minutes.

The invention also relates to a kit for enhancing the delivery of a medicant to an abnormal brain region and/or to a malignant tumor. The kit is an assemblage of materials or components, including a potassium channel activator, other than bradykinin or a bradykinin analog, as described above. In addition, the kit contains instructions for using the potassium channel activator to enhance the permeability of abnormal microvascular, including neomicrovasculature, to a medicant in general, or alternatively, to a particular medicant. Optionally, the kit also contains other components, such as a particular medicant in any pharmaceutically acceptable formulation, or paraphernalia for injection or infusion, for example syringes, infusion lines, clamps, and/or infusion bags/bottles, which can contain a pharmaceutically acceptable infusible formulation of the potassium channel activator with or without a particular medicant also contained therein. The materials or components assembled in the kit can be provided to the practitioner stored in any convenient and suitable ways that preserve their operability and utility. For example the components can be in a dissolved, dehydrated, or lyophilized form; they can be provided at room, refrigerated or frozen temperatures.

The foregoing descriptions of the methods and kits of the present invention are illustrative and by no means exhaustive. The invention will now be described in greater detail by reference to the following non-limiting examples.

### EXAMPLES

### Example 1

### Methods

Malignant Cell Line and Tumor Implantation. A rat glioma cell line, RG2, was used for implantation of experimental brain tumors in female Wistar rats. The techniques for RG2 cell propagation and maintenance in tissue culture have been described (Sugita, M. and Black, K. L., Cyclic GMP-specific phosphodiesterase inhibition and intracarotid bradykinin infusion enhances permeability into brain tumors, Cancer Res. 58(5): 914-20 [1998]; Inamura et al. [1994]; Nakano, S. et al., Increased brain tumor microvessel permeability after intracarotid bradykinin infusion is mediated by nitric oxide, Cancer Res. 56(17): 4027-31 [1996]). Briefly, RG2 cells derived from a rat glioma are kept frozen until use, then are thawed and maintained in a monolayer culture in F12 medium with 10% calf serum.

The Wistar rats (approximately 140-160 g body weight) were anesthetized with intra-peritoneal ketamine (50 mg/kg), and glial cells (1×10⁵) were implanted into the right hemisphere, but not the contralateral hemisphere, by intracerebral injection suspended in 5 μL F12 medium (1-2% methylcellulose) by a Hamilton syringe. The implantation coordinates were 3-mm lateral to the bregma and 4.5 mm deep to the dural surface.

Intracarotid Infusion of Potassium Channel Activators. Seven days after implantation of RG2 cells, the rats were anesthetized as described above and prepared for permeability studies. Animals were infused with either NS-1619 (a selective large conductance Ca²⁺-activated K⁺ channel activator; RBI, Natick, Mass.) or minoxidil sulfate (a K_{ATP} channel activator) into the right carotid artery at a dose rate of 5.3 μg kg⁻¹min⁻¹ (in 53.3 μL/min) for 15 minutes, in an infusion vehicle of PBS, pH 7.4; 5% (v/v) ethanol. Ethanol (25% [v/v]) was used to dissolve the potassium channel activators before dilution in PBS. For blood volume studies, 5 and 14 minutes after the start of the intracarotid infusion of potassium channel activator compounds, [¹⁴C] Dextran (100 μCi/kg; Dupont-New England Nuclear Co., Boston, Mass.) was injected as an intravenous bolus and maintained for 1 minute and 10 minutes to obtain two different time points. For regional permeability studies, 5 minutes after the start of the intracarotid infusion of vasoactive compounds, 100 μCi/kg of [¹⁴C]α-aminoisobutyric acid (Dupont-New England Nuclear Co., Boston, Mass.) was injected as an intravenous bolus into the right femoral vein. A peristaltic withdrawal pump was used to withdraw femoral arterial blood at a constant rate of 0.083 mL/min immediately after the injection of the tracer to determine serum radioactivity. Fifteen minutes after the intracarotid infusion, rat decapitated and the brain rapidly removed and frozen for quantitative autoradiography.

In permeability studies with nitric oxide (NO) donors, intracarotid infusion of NO donors was done as described above at a dose rate of 2.66 μg min⁻¹kg⁻¹ for 15 minute. Nitric oxide donors included sodium 2-(N,N-diethylamino)-diazenolate-2-oxide (DEA/NO), a nitric oxide-donor with a half-life of 2.1 min or PAPA/NO ([Z]-1-[N-[3-Amino propyo]-N-[N-propyo amino]diazen-1-ium-1,2-diolate; (Alexis Corp.), a nitric oxide-donor with a half-life of 15 minutes. DEA/NO or PAPA/NO were dissolved in PBS and administered to RG2 glioma-bearing rats to determine permeability (Kᵢ) of [¹⁴C]-AIB without affecting the physiological parameters. [¹⁴C]-AIB was administered intravenously as a bolus and Kᵢ determined. Physiological parameters were monitored during the experiments.

Unidirectional Transport Constant (Kᵢ). The unidirectional transfer constant Kᵢ for [¹⁴C]α-aminoisobutyric acid (AIB) was measured in normal tissue and tumor tissue as an indicator of permeability across the blood-tumor and blood-brain barriers. Quantitative autoradiography was used to obtain Kᵢ values (μL g⁻¹, min⁻¹). The initial rate for blood-to-brain transfer was calculated using a previously described equation. (Ohno, K, et al., Lower limits of cerebrovascular permeability to nonelectrolytes in the conscious rat, Am. J. Physiol. 235(3):H299-307, [1978]; Inamura, T., et al., Bradykinin selectively opens blood-tumor barrier in experimental brain tumors, J. Cereb. Flow Metab. 14(5): 862-70 [1994]). Quantitative data were analyzed using, two group t-test and two-group Fishers-exact test of equal proportions or equal means (equal numbers) at 90% power requires a minimum of 6 animals in each group to achieve statistical significance. Multiple treatment groups were compared with control group by ANOVA and P values determined by post-hoc Bonferroni test.

Dose-dependence studies. NS-1619 was dissolved in 25% ethanol and diluted with PBS to obtain various concentrations for infusion. NS-1619 was administered by intracarotid infusion (dose rates: 0, 13, 26.5, 53, 80, 100 and 110 μg kg⁻¹min⁻¹; all at 53.3 μL/min) to RG2 glioma-bearing rats to determine a dose that produces increased permeability (Kᵢ) of [¹⁴C]-AIB, which was administered intravenously. Kᵢ was determined as described above. Physiological parameters were monitored during the experiments.

Inhibition studies. Since NS-1619 increased permeability, the specificity of its effect was examined using the specific K_{Ca} channel inhibitor, iberiotoxin (RBI, Natick, Mass.; ([IBTX] was diluted in saline to a final stock concentration of 100 g/L). IBTX (2.3 μg kg⁻¹min⁻¹) was co-infused with NS-1619 (26.5 μg kg⁻¹min⁻¹) to block K_{Ca} channel-induced permeability in abnormal capillaries in the RG2 glioma rat model. Seventeen rats were used for these studies (3 vehicle-only control [i.e., PBS+5% (v/v) ethanol]; 3 IBTX; 8 NS-1619; 3 NS-1619+IBTX).

In other experiments, IBTX (0.2 μg min⁻¹kg⁻¹ for 15 min) was co-infused with the nitric oxide donor DEA/NO (2.66 μg min⁻¹kg⁻¹ for 15 min) to block K_{Ca} channel-induced permeability in abnormal capillaries in the RG2 glioma rat model. Thirteen rats were used for these studies (4 vehicle-only control; 3 DEA/NO, 3 PAPA/NO, and 3 DEAINO+IBTX).

In other in vivo permeability experiments using Wistar rats harboring intracranial RG2 tumors, as described above, the unidirectional transport constant (Kᵢ) for [¹⁴C]-AIB to the tumor center and tissue surrounding tumor was determined after intracarotid infusion of bradykinin (10 μg min⁻¹kg⁻¹ for 15 min) or minoxidil sulfate (26.6 μg min⁻¹kg⁻¹ for 15 min) or co-infusion of bradykinin with minoxidil sulfate for 15 min. Physiological parameters were monitored during the experiments. The selective K_{ATP} inhibitor, glibenclamide (13.3 μg min⁻¹kg⁻¹ for 15 min) was used to determine whether inhibition of K_{ATP} channel blocks bradykinin-inducible or minoxidil sulfate-inducible increases in blood-tumor barrier permeability.

In other experiments, RG2 glioma cells were implanted into the right hemisphere of rat brains as described above. Seven days after implantation, regional permeability study was performed by intracarotid infusion of octobromo-cyclic GMP (8Br-cGMP, 16.7 μg kg⁻¹min⁻¹), an activator of cyclic GMP-dependent protein kinase (PKG), without or with PKG inhibitors (KT5823, 6.77 μg kg⁻¹min⁻¹; or Rp-pCPT-cGMP, 60 μg kg⁻¹min⁻¹). Saline or 2% DMSO was infused into rats in a control group. At the termination of the study, rats were killed under anesthesia, and brains were removed for measurement of Kᵢ for [¹⁴C]-AIB (μL g⁻¹ min⁻¹). Western blot analysis was also used to compare PKG expression levels in normal brain and tumor tissue.

Quantitative autoradiography and calculations of Kᵢ for [¹⁴C]-AIB were performed as described above, according to Ohno et al. (1978).

Immunohistochemical analysis for K_{Ca} channels. Brain sections (12 μm thick) obtained from the permeability studies were incubated with 1:100 dilution of affinity-purified K_{Ca} channel antibody (Alomone Labs, Jerusalem, Israel) for 1 hour, and biotinylated horse anti-mouse immunoglobin (Vector Laboratories, Burlingame, Calif.) for 30 minutes. After washing 3 times with PBS, the peroxidase sites were visualized using an avidin:biotinylated enzyme complex (ABC) kit.

Transmission Electron Microscopy (TEM). Rat brain tissue was prepared for TEM analysis by the following procedures. After experiments involving intracarotid infusion of potassium channel activators, inhibitors, horse radish peroxidase (HRP), and/or vehicle control buffers, rats were infusion-fixed from the heart first with PBS (50-100 mL) followed with 1.0% glutaraldehyde (250 mL). The brain tissue was cut cronally to expose the tumor region, and the region of interest was selected, sliced into small pieces of about 1 mm thickness, and was immersion-fixed in 1.0% glutaraldehyde, at 4° C. for 2 hours. The sample was rinsed in 5% sucrose/0.1 M PBS at 4° C., overnight, with continuous shaking and changes of the solution. The samples then were immersion-fixed with 1% OsO4 at 4° C. for 2 hours with continuous shaking. The sample was then dehydrated with an increasing concentration (50-100%) of ethanol at 4° C. for 15 minutes with constant shaking and frequent changes of solution. Samples were infiltrated with propylene oxide, and then with epon at room temperature with shaking. Embedding was done at 60° C. over 48 hours. Semi-thin sections (about 30-50 μm thick) were taken with a microtome equipped with a diamond-edge blade. Finally, ultrathin sections (about 1-10 nm thick) were prepared with a microtome and stained with uranyl acetate for 45-60 minutes, lead citrate for 5 minutes and processed for TEM analysis.

Ten capillaries in each treatment group were selected and photographed at low magnification (total 90 capillaries). The basic morphology of the capillaries was examined by way of measuring and calculating the following parameters which represent the basic profile of the capillary: abluminal circumference, luminal circumference, total area of the capillary (excluding nuclei and vacuoles), and mean thickness of the capillary.

In a separate study, the density of the vesicles was determined for three vessels selected from each rat (each treatment group contained 5-6 rats) by taking from each blood vessel four electron micrographs as a test zone at high magnification. The test zones were selected randomly, such as at 3, 6, 9, and 12 o'clock on the EM screen. The areas of the test zones were measured, and the number of vesicles was counted by a person who was not aware of the background of the micrographs. Vesicular density was expressed as the number of vesicles per square micrometer of cytoplasm. The proportion of the total vesicular area to the cytoplasmic area was also determined using the same micrographs: total vesicular area was measured and the proportion to the cytoplasm which included those vesicles was calculated and expressed as percentage. The mean diameter of the vesicles was also calculated.

Morphological changes resulting from the treatments were examined by selecting six tight junctions from each rat (five rats were in each treatment group for a total 270 tight junctions). The tight junctions were photographed at high magnification. The analysis of tight junction was by comparing “cleft index” and “cleft area index” for each tight junction. The cleft index was expressed by the following formula: (length of unfused segments)÷(length of junctions). (Stewart, 1987). The cleft area index was expressed by the formula: (area of unfused segments)÷(length of junction).

Temporary Middle Cerebral Artery (MCA) occlusion. MCA occlusion was carried out as described by Liu, Y. et al., The time course of glucose metabolism in rat cerebral ischemia with middle cerebral artery occlusion-reperfusion model and the effect of MK-80, Neurological Research 18 (6): 505-508 (1996) with slight modifications. Briefly, the right MCA was occluded temporarily with a silicone rubber cylinder inserted from the bifurcation of the intracarotid artery after ligation of ipsilateral common and external carotid arteries. The cylinder is made of 4-0 nylon 17 mm long surgical thread, coated with silicone mixed with a hardener to grade the diameter of the distal 5 mm to 0.25-0.30 mm. The thread was inserted into the intracarotid artery through the external carotid artery near the bifurcation and ligated at the point of insertion. The silicone rubber cylinder reached the proximal portion of the anterior cerebral artery. The origin of the right MCA and posterior-communicating artery were occluded by the thread. Following the surgery, animals were immobilized by means of loose fitting Pasteur casts and allowed to recover from anesthesia. The thread was pulled out from the intracarotid artery after 1 hour or 2 hours of ischemia to allow reperfusion of the ischemic tissue with blood. Potassium channel activator(s) and/or inhibitor(s) were injected by intracarotid infusion after 45 minutes of reperfusion for an additional 15 minutes.

In some experiments, Evans blue dye was injected intravenously after 50 minutes of reperfusion (five minutes after commencing administration of potassium channel activator and/or inhibitor). Ten minutes after injection of Evans blue, the rat was perfused with 200 mL PBS through the heart to wash off excess Evans blue dye from the brain microvessels.

### Example 2

### Results

Potassium Channel Activators Selectively Increase Transport Across the Blood-Tumor Barrier.

When Wistar rats bearing implanted glioma cells were infused with either NS-1619 or minoxidil sulfate, at 7.5 μg kg⁻¹min⁻¹ for 15 minutes, the unidirectional transport constant K for [¹⁴C]α-aminoisobutyric acid (AIB) was significantly increased by NS-1619 and minoxidil sulfate with respect to transport across the neovasculature forming the blood-tumor barrier, but not with respect to transport across normal brain microvasculature (data not shown). These results demonstrated that activation of potassium calcium channels selectively increases the permeability of molecules across the capillaries of solid malignant tumors compared to capillaries supplying normal brain tissue.

Increasing the dose of NS-1619 resulted in an increase in the unidirectional transfer constant Kᵢ for [¹⁴C]α-aminoisobutyric acid in RG2 glioma capillaries in a dose-dependent manner (data not shown). At higher doses (100 and 110 μg/kg/min) a significant drop in the arterial blood pressure of the rats was observed.

The specificity of this effect was demonstrated, because the ability of NS-1619 to increase the unidirectional transfer constant Kᵢ for [¹⁴C]α-aminoisobutyric acid was inhibited by the K_{Ca}-channel-specific inhibitor iberiotoxin (IBTX; data not shown). The Kᵢ was determined in RG2 tumor-bearing rats using [¹⁴C]-AIB with NS-1619 (26.5 μg min⁻¹kg⁻¹) with or without IBTX (2.3 μg kg⁻¹min⁻¹; n=3), for 15 minutes. Increase of Kᵢ in response to NS-1619 infusion (n=8; ** P<0.001 compared with PBS with or without 5% ethanol) was attenuated by IBTX co-treatment. IBTX alone at the dose investigated did not affect the brain-tumor barrier permeability of abnormal capillaries. However, IBTX significantly (n=3, ** P<0.001 compared with NS-1619-treated group) decreased NS-1619-induced increase of permeability (Kᵢ), indicating a potassium channel-specific effect. Controls receiving PBS plus 5% ethanol were indistinguishable from controls receiving PBS minus ethanol.

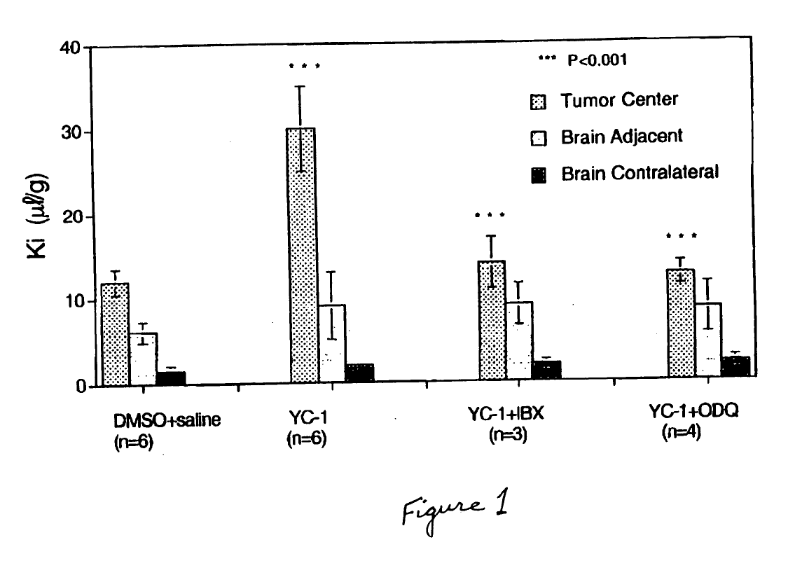

A comparable increase in blood-tumor barrier permeability was obtained when the soluble guanylyl cyclase activator YC-1 was administered (FIG. 1). IBTX and a selective inhibitor of soluble guanylyl cyclase, 1H-[1,2,4]oxadiazolo[4,3-a]quinozalin-1-one (ODQ), attenuated the enhancement of Kᵢ by YC-1, showing it was K_{Ca}-mediated.

Infusion of minoxidil sulfate resulted in a significant increase in Kᵢ (28.3±6.0 μL/g/min; P<0.001), compared to the vehicle control group (FIG. 7). This increase in blood-tumor permeability was attenuated by the K_{ATP} channel inhibitor glibenclamide, which significantly attenuated (K_{i=12.7±2.0} μL/g/min) the effect of minoxidil sulfate. Thus, the Kᵢ increases with minoxidil sulfate were due to activation of K_{ATP} channels, which selectively enhanced blood-tumor barrier permeability. The increase in permeability resulting from treatment with minoxidil sulfate was comparable to that caused by the K_{Ca} activator bradykinin (Kᵢ=34±8.0 μL/g/min), and a significant additive effect on permeability (Kᵢ=42±6 μL/g/min; P<0.05) was achieved with combination treatment with bradykinin and minoxidil sulfate as compared to individual treatment groups (FIG. 7).

The co-infusion with glibenclamide failed to block bradykinin-inducible Kᵢ increase (FIG. 8) is consistent with other studies showing bradykinin does not modulate K_{ATP} channels. However, the K_{Ca} channel antagonist, IBTX, significantly attenuated bradykinins effect (Kᵢ=17.7±6 μL/g/min; P<0.01) indicating that the Kᵢ increase was due to activation of K_{Ca} and not K_{ATP} channels by bradykinin (FIG. 8).

Minoxidil sulfate-induced effect was significantly attenuated by glibenclamide (Kᵢ=12.7±2 μL/g/min; P<0.001; FIG. 9). However, this effect was not blocked by IBTX indicating that K_{ATP} channels mediate minoxidil sulfate-induced permeability. These results imply independent pathways for blood-tumor barrier permeability, one regulated by K_{Ca} and the other by K_{ATP} channels in the microcapillary of the brain tumor.

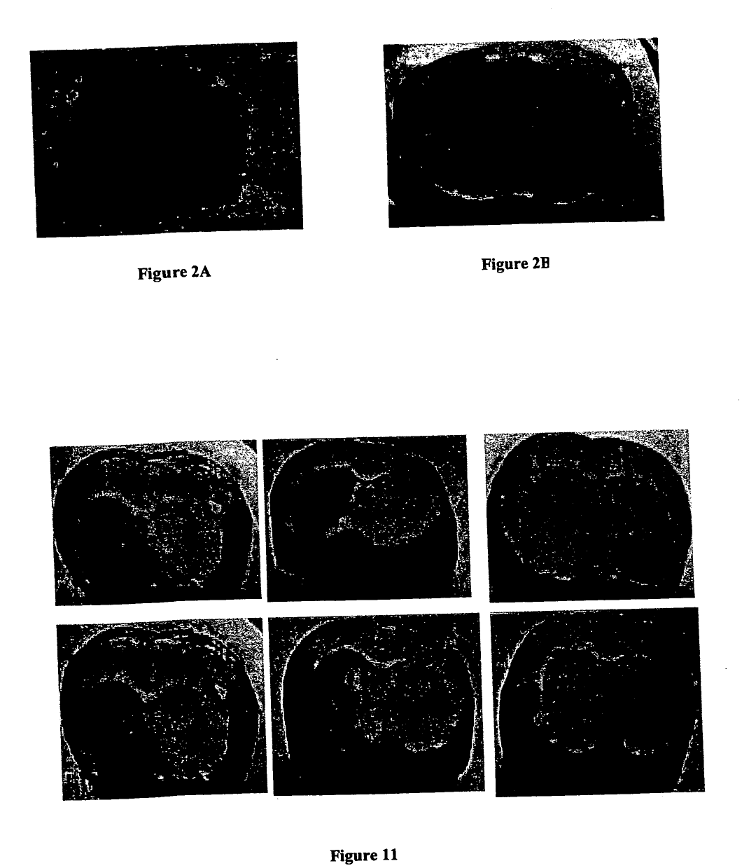

Nitric oxide donors also increased the permeability of the blood-tumor barrier. FIG. 2 shows that RG2 glioma tissue was stained in vivo by Evans blue stain (MW 960.82) in DEA/NO-treated Wistar rats (FIG. 2B) compared to PBS-treated controls (FIG. 2A). These results were comparable to the results obtained by in vivo staining by Evans blue using NS-1619 or YC-1 (data not shown).

FIG. 3 shows that Kᵢ increased markedly in response to DEA/NO and PAPA/NO (Kᵢ=32±9.0 μL g⁻¹ min⁻¹ and 36.4±4.0 μL g⁻¹min⁻¹, respectively) compared to controls (saline). However, co-treatment with IBTX attenuated the effect of infusions of DEA/NO (Kᵢ=15.3±2.3 μL g⁻¹min⁻¹; P<0.001) or PAPA/NO (Kᵢ=15.3±1.7 μL g⁻¹min⁻¹; P<0.001) and, showing that the effect was K_{Ca}-mediated. IBTX alone at the dose investigated did not affect the brain-tumor barrier permeability of abnormal capillaries.

Western blots revealed PKG expression in both normal brain and tumor tissue. PKG activator 8Br-cGMP increased Kᵢ values compared with vehicle-only controls (Kᵢ=22.1±6.6 μL g⁻¹min⁻¹ versus 14.2±5.1 μg⁻¹min⁻¹, p<0.05). These results imply that endogenous production of cGMP, which activates PKG, selectively increases blood-tumor barrier permeability, presumably by PKG-mediated activation of K_{Ca}.

Immunohistochemical Analysis Shows Potassium Channels Are More Abundant in Neovasculature and Malignant Cells Compared to Normal Tissue.

K_{Ca} channel protein was immunolocalized using a specific antibody as described above. Immunohistochemical analysis showed that K_{Ca} channels were selectively increased in tumor tissue and tumor capillaries in RG2 bearing rat brain sections, compared to sections of normal contralateral tissue. These immunohistochemical results are consistent with the permeability data in which activation of K_{Ca} channel by NS-1619 selectively opened the blood-tumor barrier.

Together, the permeability and immunohistochemical data demonstrate that compounds that activate potassium channels can be used to selectively increase delivery of anti-tumor compounds to malignant tumor tissue.

Transmission Electron Microscopic studies. Transmission electron microscopic (TEM) studies revealed accelerated formation and movement of pinocytotic vesicles along the luminal-abluminal axis of capillary endothelium, as well as tumor cells, following intracarotid infusion of K_{Ca} activators NS-1619 and bradykinin, compared to a PBS control (FIG. 4). Quantitative analysis showed that both bradykinin and NS-1619 significantly increased the number of vesicles per unit surface area of nucleus-free cytoplasm, compared to the PBS control. The pinocytotic vesicles had an average diameter of 75-80 nm. Invagination of luminal membrane of tumor capillary endothelial cells gives rise to arrays of pinocytotic vesicles, which migrate along a luminal-abluminal axis in endothelial cytoplasm (E). These vesicles dock and fuse with the basement membrane, releasing their contents on abluminal side of endothelial cell membrane. Cells from the treatment group appear to have intact basal membrane (BM) and endothelial tight junctions. Transport pinocytotic vesicles (PV) are present near luminal (L) as well as abluminal (Ab) areas (FIG. 4). Cells from gliomas after infusion of either bradykinin or NS-1619 have intact basal membrane and endothelial tight junctions (TJ). (E.g., FIG. 5). Additional TEM studies revealed the presence of electron dense horse radish peroxidase (HRP; protein marker with a molecular weight of about 40,000) within the arrays of pinocytotic vesicles in endothelial cells of brain tumor capillaries and showed HRP being transported from the luminal to the abluminal area of the endothelium to the tumor cells after the K_{Ca} activator bradykinin was co-infused with HRP into rats with RG2 tumors. (FIGS. 5 and 6). This evidence of HRP-laden transport vesicles implies that transendothelial vesicular transport is a significant cellular mechanism for drug delivery to the tumor cells.

Additional TEM studies with tumor-bearing rats infused with the potassium channel activator bradykinin, and intravenously injected with the tracer horseradish peroxidase (HRP), revealed the presence of electron dense HRP within the arrays of pinocytotic vesicles in endothelial cells of brain tumor capillaries (FIG. 5; P=plasma membrane). Following bradykinin infusion there was an increase in the number of HRP-laden vesicles (observed in the tumor cells compared to PBS-treated tumor cells FIG. 6; arrows), implying that vesicular transport is a primary cellular mechanism for drug delivery to the tumor cells, once the drug molecules cross the capillary endothelium into the brain tumor mass.

No significant difference was seen in any of the examined morphological parameters between the vehicle control and the bradykinin (BK) infusion group, but there was a significant difference in these parameters in comparing the different kinds of tissue examined. Abluminal and luminal circumference, area of the capillary, and mean thickness of the capillary of RG2 tumor and C6 tumor were significantly larger than those of normal brain basal ganglia. However, a significant increase in density and proportion of vesicle area in the basal ganglia was observed inbradykinin-treated rats in RG2 and C6 tumor area as compared to the untreated rats (Table 1).

TABLE 1
Density and proportion of the area of vesicles to endothelial cytoplasm.

| | | Density of the vesicles | Total area of |
|---|---|---|---|
| Tissue^{a} | Infusion^{aa} | (number/m2)^{b} | the vesicles (%)^{c} |
| BG | PBS | 2.02 ± 0.35 (n = 5) | 0.65 ± 0.26 (n = 6) |
| | BK | 1.75 ± 0.90 (n = 5) | 0.54 ± 0.35 (n = 6) |
| RG2 tumor | PBS | 3.29 ± 0.66 (n = 5) | 1.30 ± 0.36 (n = 6) |
| | BK | 9.88 ± 2.93 d (n = 6) | 3.71 ± 0.96d (n = 6) |
| C6 tumor | PBS | 5.46 ± 3.29 (n = 5) | 1.34 ± 0.57 (n = 6) |
| | BK | 11.90 ± 2.40d (n = 5) | 4.31 ± 1.34^{d} (n = 6) |

^{a}BG = basal ganglia.
^{aa}PBS = phosphate buffer saline vehicle control; BK = PBS + bradykinin.
^{b}Values are means SD; n = number of rats.
^{c}Values are means SD; n = number of capillaries.
^{d}Significant difference from saline control group at p < 0.01

Potassium Channel Activators Increase Permeability in Abnormal Brain Capillaries of Ischemic Brain Region.

In preliminary MCA occlusion/reperfusion studies it was shown that the K_{Ca} activator bradykinin increases permeability in infarct tissue after transient-ischemia. NOS activity was significantly elevated in transient ischemia-reperfusion rat models (data not shown), corresponding to the ability of bradykinin to increase permeability in ischemic capillaries (FIG. 10B). As shown in FIG. 10B, the permeability of microvasculature in ischemic brain regions exhibited enhanced responsiveness to potassium channel activator after two hours of MCA occlusion, compared to normal brain microvasculature (i.e., contralateral tissue). In contrast, FIG. 10A shows that the permeability of brain microvasculature was not responsive to a potassium channel activator after only one hour of MCA occlusion. This indicates that properties of the microvasculature are changed by prolonged ischemia as the tissue becomes increasingly abnormal.

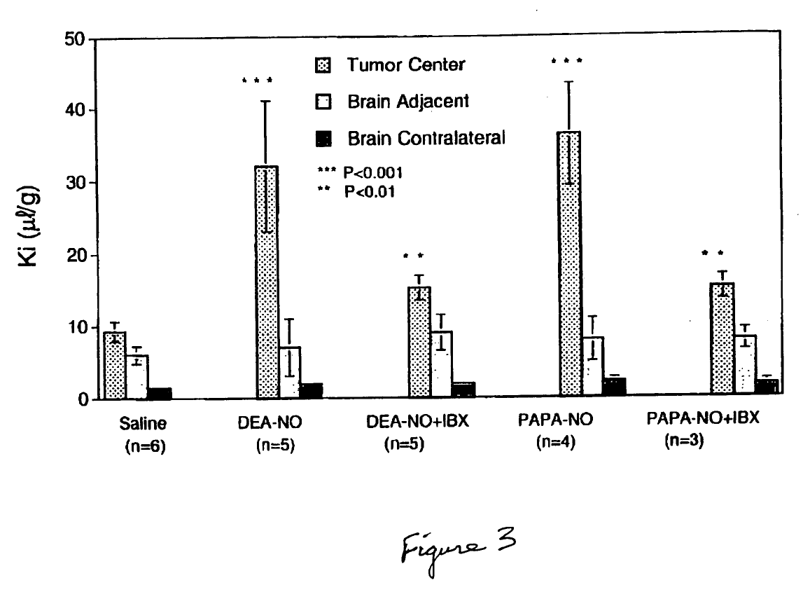

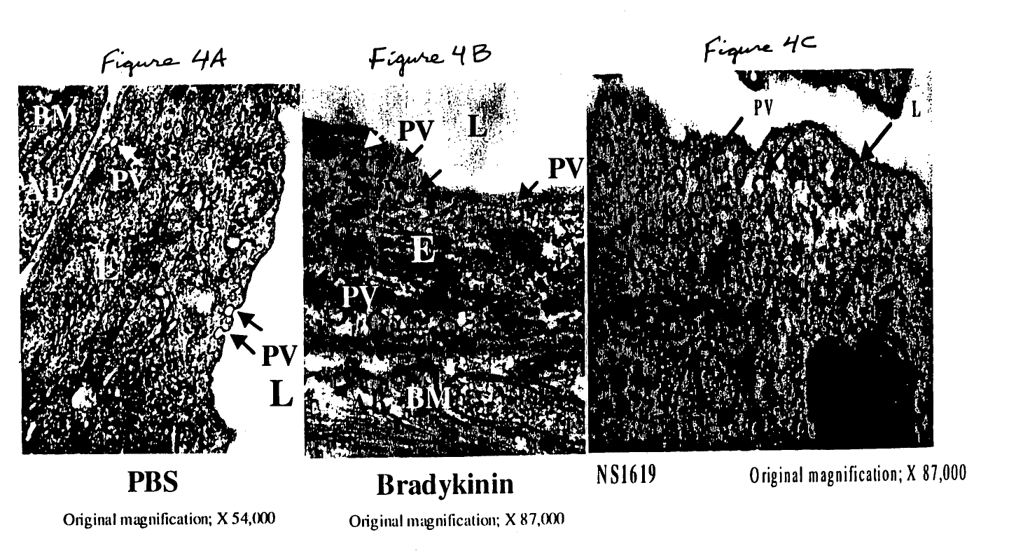

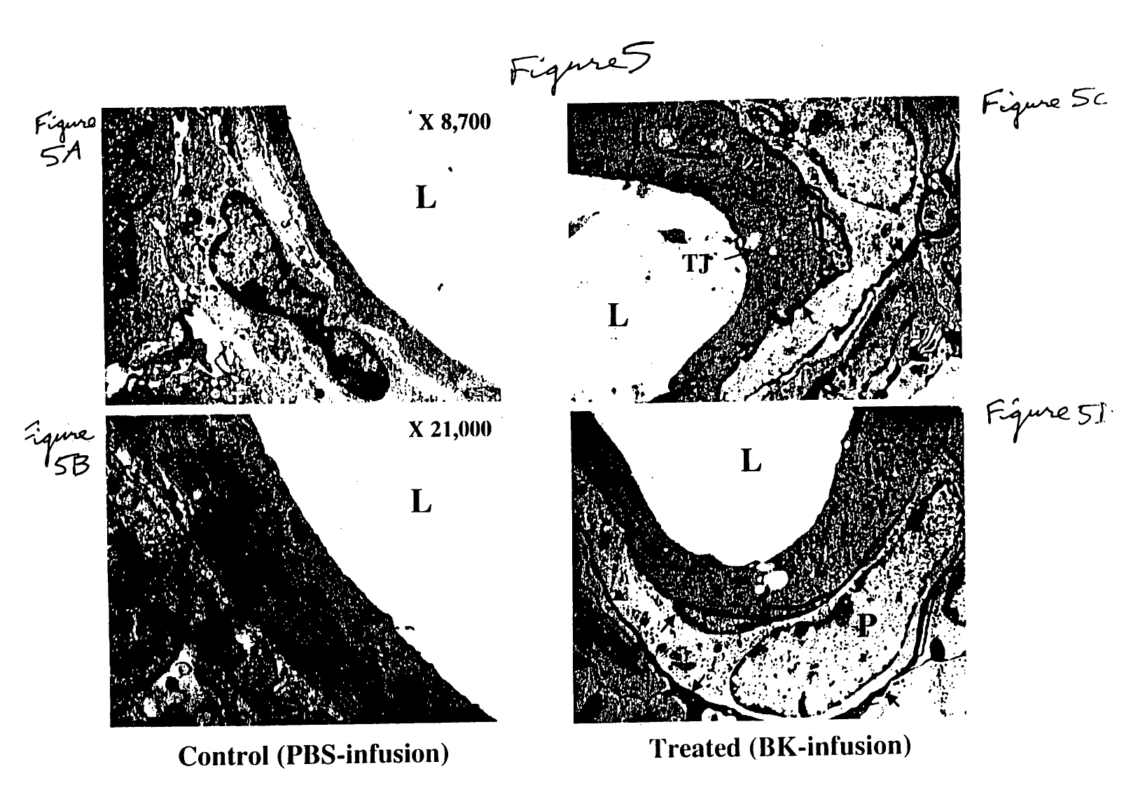

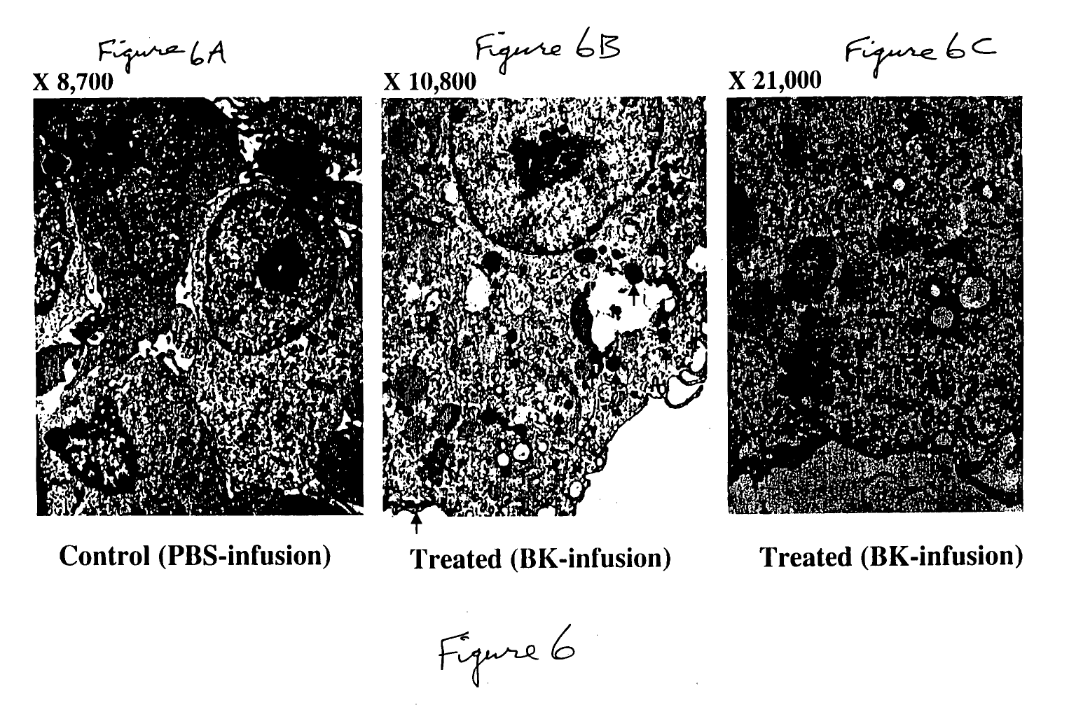

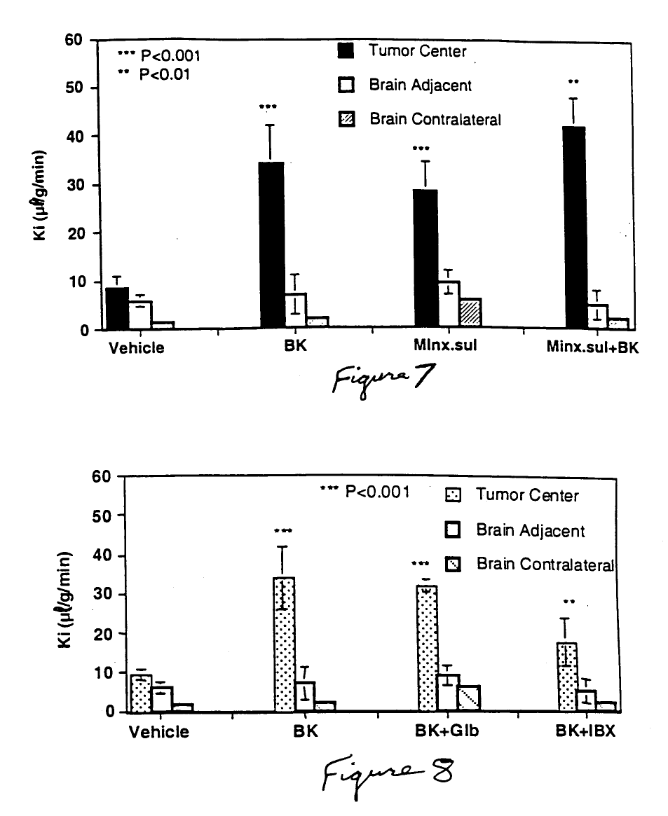

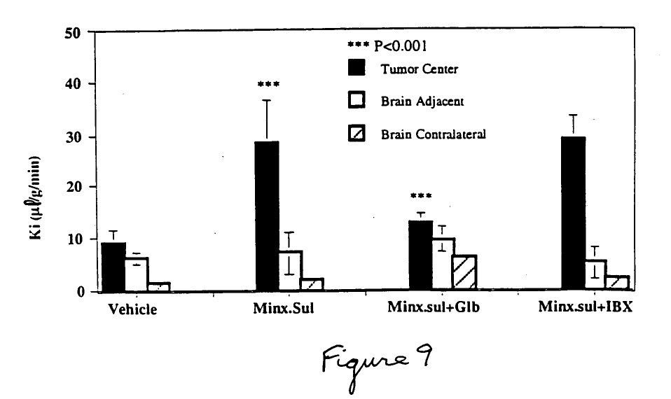

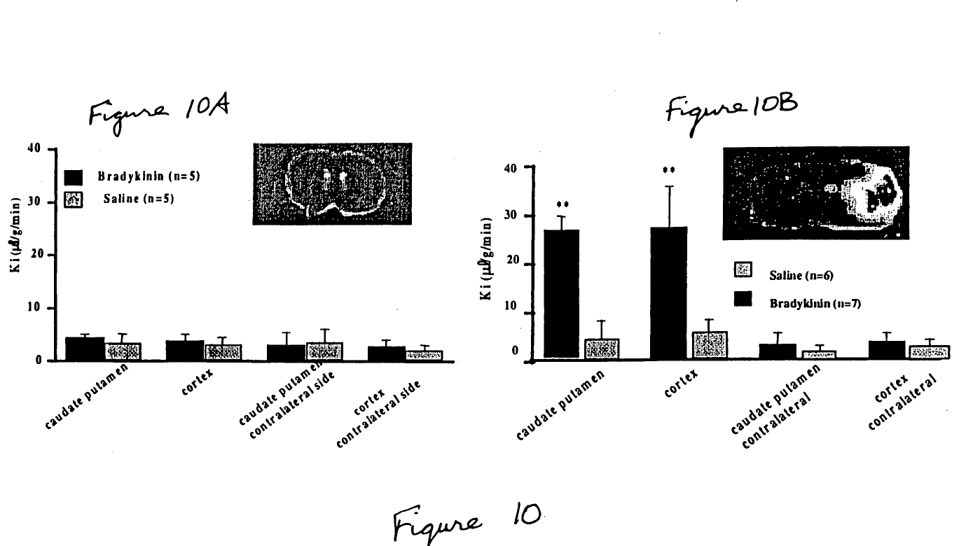

Using the ischemia-reperfusion rat model, it was directly observed that enhanced permeability to Evans blue dye was regulated, by potassium channels in ischemic brain regions. In FIG. 11, coronal brain sections show Evans blue (EB) staining within the tumor tissue. The uptake of Evans blue dye is a known qualitative measure of blood-brain barrier/blood-tumor barrier permeability. Rats subjected to 2 hour of MCA occlusion followed by 1 hour of reperfusion did not show an increase in permeability in ischemic areas after PBS vehicle infusion (FIG. 11). In contrast, rats subjected to similar ischemic conditions followed by intra arterial infusion of activators of K_{Ca} channels, for example bradykinin or NS-1619 (FIG. 11) or nitric oxide donors (data not shown), enhanced permeability to the dye. The increased permeability to the dye produced by the potassium channel activators was diminished by co-infusion with the specific K_{Ca} inhibitor IBTX, indicating the specific involvement of K_{Ca} (FIG. 11).

1-161. (canceled)

162. A method of delivering a medicant to an abnormal brain region in a mammalian subject, comprising: administering to a mammalian subject having an abnormal brain region a direct agonist of an ATP-sensitive potassium channel, under conditions and in an amount sufficient to increase the permeability to the medicant of a capillary or arteriole delivering blood to cells of the abnormal brain region; and administering to the subject, simultaneously or substantially simultaneously with the direct agonist the medicant, so that the medicant is delivered selectively to the cells of the abnormal brain region compared to normal brain regions.

163. The method of claim 162, wherein the abnormal brain region is a region of brain tissue physiologically affected by injury, trauma, infection, stroke or ischemia.

164. The method of claim 162, wherein the abnormal brain region is a region of tumor tissue.

165. The method of claim 162, wherein the mammal is a human.

166. The method of claim 162, wherein the medicant is a therapeutic cytotoxic agent or an anticancer chemotherapeutic agent.

167. The method of claim 162, wherein the direct agonist is administered by intravenous or intra-arterial infusion or injection.

168. The method of claim 162, wherein administering the direct agonist is administered by intracarotid infusion or injection.

169. The method of claim 162, wherein the direct agonist is administered to the mammalian subject by intravenous infusion.

170. The method of claim 162, wherein direct agonist is administered to the mammalian subject in an amount from about 0.075 to 1500 micrograms per kilogram body mass.

171. The method of claim 170, wherein the direct agonist is administered to the subject in an amount from about 0.075 to 150 micrograms per kilogram body mass.

172. The method of claim 162, wherein the direct agonist is administered to the mammalian subject at a dose rate of about 0.075 to about 100 μg kg⁻¹min⁻¹ for up to about 30 minutes.

173. The method of claim 172, wherein the direct agonist is administered to the mammalian subject in a bolus injection.

174. A method of delivering a medicant to a malignant tumor in a mammalian subject, comprising: administering to a mammalian subject having a malignant tumor a direct agonist of an ATP-sensitive potassium channel, under conditions and in an amount sufficient to increase the permeability to the medicant of a capillary or arteriole delivering blood to cells of the malignant tumor; and administering to the subject, simultaneously or substantially simultaneously with the direct agonist the medicant, so that the medicant is delivered selectively to the malignant cells compared to non-malignant cells.

175. The method of claim 174 wherein the malignant tumor is a glioma, glioblastoma, oligodendroglioma, astrocytoma, ependymoma, primitive neuroectodetermal tumor, atypical meningioma, malignant meningioma, neuroblastoma, sarcoma, melanoma, lymphoma or carcinoma.

176. The method of claim 174, wherein the malignant tumor is contained in the skull, brain, spine, thorax, lung, peritoneum, prostate, ovary, uterus, breast, stomach, liver, bowel, colon, rectum, bone, lymphatic system, or skin, of said subject.

177. The method of claim 174, wherein said mammal is a human.

178. The method of claim 174, wherein the medicant is a therapeutic cytotoxic agent.

179. The method of claim 174, wherein the direct agonist is administered by intravenous or intra-arterial infusion or injection.

180. The method of claim 174, wherein the direct agonist is administered by intracarotid infusion or injection.

181. The method of claim 174, wherein the direct agonist is administered to the mammalian subject by intravenous infusion.

182. The method of claim 174, wherein the direct agonist is administered to the mammalian subject in an amount from about 0.075 to 1500 micrograms per kilogram body mass.

183. The method of claim 182, wherein the direct agonist is administered to the subject in an amount from about 0.075 to 150 micrograms per kilogram body mass.

184. The method of claim 174, wherein the direct agonist is administered to the mammalian subject at a dose rate of about 0.075 to about 100 μg kg⁻¹ min⁻¹ for up to about 30 minutes.

185. The method of claim 184, wherein the direct agonist is administered by bolus injection.

186. A pharmaceutical composition comprising a combination of a direct agonist of an ATP-sensitive potassium channel, formulated in a pharmaceutically acceptable solution together with a medicant selected from the group consisting of therapeutic cytotoxic agents or anticancer chemotherapeutic agents for delivery by intravascular infusion or injection into a mammal.

187. The pharmaceutical composition of claim 186, wherein the direct agonist is present in an amount of about 0.075 to 1500 micrograms per kilogram body mass.

188. The pharmaceutical composition of claim 186, wherein the direct agonist is present in an amount of about 0.075 to 150 micrograms per kilogram body mass.

189. The pharmaceutical composition of claim 186, further comprising a buffer solution pharmaceutically acceptable for intravascular infusion into an animal.

190. The pharmaceutical composition of claim 186, wherein the buffer solution is phosphate buffered saline.

191. A kit for enhancing the delivery of a medicant to an abnormal brain region and/or to a malignant tumor, comprising: a direct agonist of an ATP-sensitive potassium channel; and instructions for using the direct agonist for enhancing the delivery of a medicant to an abnormal brain region or to a malignant tumor.

192. The method of claim 162, wherein the direct agonist is minoxidil.

193. The method of claim 162, wherein the direct agonist is minoxidil sulfate.

194. The method of claim 162, wherein the direct agonist is cromakalim.

195. The method of claim 162, wherein the direct agonist is levcromakalim.

196. The method of claim 162, wherein the direct agonist is pinacidil.

197. The method of claim 162, wherein the direct agonist is diazoxide.

198. The method of claim 162, wherein the abnormal brain region is a region of brain tissue physiologically affected by stroke.

199. The method of claim 162, wherein the abnormal brain region is a region of brain tissue physiologically affected by ischemia.

200. The method of claim 162, wherein the abnormal brain region is a region of brain tissue physiologically affected by injury or trauma.

201. The method of claim 162, wherein the abnormal brain region is a region of brain tissue physiologically affected by infection.

202. The method of claim 162, wherein the abnormal brain region is a region of benign tumor tissue.

203. The method of claim 162, wherein the abnormal brain region is a region of malignant tumor tissue.

204. The method of claim 162, wherein the abnormal brain region includes a glioma, glioblastoma, oligodendroglioma, astrocytoma, ependymoma, primitive neuroectodermal tumor, atypical meningioma, malignant meningioma, neuroblastoma, sarcoma, melanoma, lymphoma, or carcinoma.

205. The method of claim 162, wherein the medicant is administered via intravenous, intramuscular, intra-arterial, or intracarotid injection or infusion.

206. The method of claim 162, wherein the agonist and the medicant are administered via intracarotid infusion or injection.

207. The method of claim 162, wherein the medicant is a protein.

208. The method of claim 162, wherein the medicant is a monoclonal antibody or antigen-binding antibody fragment.

209. The method of claim 162, wherein the medicant is a cytokine, cytokine antagonist, or cytokine agonist.

210. The method of claim 162, wherein the medicant is an interferon.

211. The method of claim 162, wherein the medicant is an interleukin.

212. The method of claim 211, wherein the interleukin is interleukin 2.

213. The method of claim 162, wherein the medicant is transforming growth factor.

214. The method of claim 213, wherein the transforming growth factor is transforming growth factor-□

215. The method of claim 162, wherein the medicant is tumor necrosis factor-□

216. The method of claim 162, wherein the medicant is an antimicrobial agent or an antibiotic.

217. The method of claim 162, wherein the medicant is an immunotoxin or immunosuppressant

218. The method of claim 162, wherein the medicant is a boron compound.

219. The method of claim 162, wherein the medicant is an ischemia-protective agent.

220. The method of claim 219, wherein the ischemia-protective agent is N-methyl-D-aspartate (NMDA) receptor antagonist.

221. The method of claim 162, wherein the medicant is an adrenergic agent.

222. The method of claim 162, wherein the medicant is an anticonvulsant.

223. The method of claim 162, wherein the medicant is an anti-trauma agent.

224. The method of claim 162, wherein the medicant is cisplatin or carboplatin.

225. The method of claim 162, wherein the medicant is methotrexate.

226. The method of claim 162, wherein the medicant is 5-flourouracil.

227. The method of claim 162, where the medicant is amphotericin.

228. The method of claim 162, wherein the medicant is daunorubicin.

229. The method of claim 162, wherein the medicant is doxorubicin.

230. The method of claim 162, wherein the medicant is vincristine.

231. The method of claim 162, wherein the medicant is vinblastine.

232. The method of claim 162, wherein the medicant is busulfan.

233. The method of claim 162, wherein the medicant is chlorambucil.

234. The method of claim 162, wherein the medicant is cyclophosphamide.

235. The method of claim 162, wherein the medicant is melphalan.

236. The method of claim 162, wherein the medicant is ethyl ethanesulfonic acid.

237. The method of claim 162, wherein the medicant is a diagnostic agent

238. The method of claim 174, wherein the direct agonist is minoxidil.

239. The method of claim 174, wherein the direct agonist is minoxidil sulfate.

240. The method of claim 174, wherein the direct agonist is cromakalim.

241. The method of claim 174, wherein the direct agonist is levcromakalim.

242. The method of claim 174, wherein the direct agonist is pinacidil.

243. The method of claim 174, wherein the direct agonist is diazoxide.

244. The method of claim 174 wherein the medicant is administered via intravenous, intramuscular, intra-arterial, or intracarotid injection or infusion.

245. The method of any claim 174, wherein the agonist and the medicant are administered via intracarotid infusion or injection.

246. The method of claim 174, wherein the medicant is a protein.

247. The method of claim 174, wherein the medicant is a monoclonal antibody or antigen-binding antibody fragment.

248. The method of claim 174, wherein the medicant is a cytokine, cytokine antagonist, or cytokine agonist.

249. The method of claim 174, wherein the medicant is an interferon.

250. The method of claim 174, wherein the medicant is an interleukin.

251. The method of claim 250, wherein the interleukin is interleukin 2.

252. The method of claim 174, wherein the medicant is transforming growth factor.

253. The method of claim 174, wherein the transforming growth factor is transforming growth factor-□

254. The method of claim 174, wherein the medicant is tumor necrosis factor-□

255. The method of claim 174 wherein the medicant is an antimicrobial agent or an antibiotic.

256. The method of claim 174, wherein the medicant is an immunotoxin or immunosuppressant.

257. The method of claim 174, wherein the medicant is a boron compound.

258. The method of claim 174, wherein the medicant is an ischemia-protective agent.

259. The method of claim 258, wherein the ischemia-protective agent is N-methyl-D-aspartate (NMDA) receptor antagonist.

260. The method of claim 174, wherein the medicant is an adrenergic agent.

261. The method of claim 174, wherein the medicant is an anticonvulsant.

262. The method of claim 174, wherein the medicant is an anti-trauma agent.

263. The method of claim 174, wherein the medicant is cisplatin or carboplatin.

264. The method of claim 174, wherein the medicant is methotrexate.

265. The method of claim 174, wherein the medicant is 5-flourouracil.

266. The method of claim 174, where the medicant is amphotericin.

267. The method of claim 174, wherein the medicant is daunorubicin.

268. The method of claim 174, wherein the medicant is doxorubicin.

269. The method of claim 174, wherein the medicant is vincristine.

270. The method of claim 174, wherein the medicant is vinblastine.

271. The method of claim 174, wherein the medicant is busulfan.

272. The method of claim 174, wherein the medicant is chlorambucil.

273. The method of claim 174, wherein the medicant is cyclophosphamide.

274. The method of claim 174, wherein the medicant is melphalan.

275. The method of claim 174, wherein the medicant is ethyl ethanesulfonic

276. The method of claim 174, wherein the medicant is a diagnostic agent

277. The pharmaceutical composition of claim 186, wherein the direct agonist is minoxidil.

278. The pharmaceutical composition of claim 186, wherein the direct agonist is minoxidil sulfate.

279. The pharmaceutical composition of claim 186, wherein the direct agonist is cromakalim.

280. The pharmaceutical composition of claim 186, wherein the direct agonist is levcromakalim.

281. The pharmaceutical composition of claim 186, wherein the direct agonist is pinacidil.

282. The pharmaceutical composition of claim 186, wherein the direct agonist is diazoxide.

283. The pharmaceutical composition of claim 186, wherein the therapeutic cytotoxic agent is cisplatin or carboplatin.

284. The pharmaceutical composition of claim 186, wherein the therapeutic cytotoxic agent is methotrexate.

285. The pharmaceutical composition of claim 186, wherein the therapeutic cytotoxic agent is 5-fluorouracil.

286. The pharmaceutical composition of claim 186, wherein the therapeutic cytotoxic agent is amphotericin.

287. The pharmaceutical composition of claim 186, wherein the anticancer chemotherapeutic agent is daunorubicin, doxorubicin, vincristine, vinblastine, busulfan, chlorambucil, cyclophosphamide, melphalan, or ethyl ethanesulfonic acid.

288. A pharmaceutical composition comprising a combination of a direct agonist of an ATP-sensitive potassium channel formulated together in a pharmaceutically acceptable solution together with a drug for delivery by intravascular infusion or injection, wherein the drug is a antimicrobial agent, antibiotic, interferon, cytokine, cytokine agonist, cytokine antagonist, monoclonal antibody, antigen-binding antibody fragment, immunotoxin, immunosuppressant, ischemia-protective agent, adrenergic agent, boron compound, anticonvulsant, anti-trauma agent or diagnostic agent.

289. A pharmaceutical composition comprising a combination of a direct agonist of an ATP-sensitive potassium channel formulated together in a pharmaceutically acceptable solution together with a drug for delivery by intravascular infusion or injection, wherein the drug is a naked DNA expression vector, protein, oligonucleotide or nucleotide analog.

290. The kit of claim 191, wherein the direct agonist is minoxidil or minoxidil sulfate.

291. The kit of claim 191, wherein the direct agonist is cromakalim, levcromakalim, pinacidil, or diazoxide.

292. The method of claim 162, wherein the medicant is a DNA expression vector or viral vector.

293. The method of claim 292, wherein the viral vector is an adenovirus-derived viral vector or herpes simplex derived viral vector.

294. The method of claim 162, wherein the medicant is an oligonuceotide or nucleotide analog.

295. The method of claim 174, wherein the medicant is a DNA expression vector or viral vector.

296. The method of claim 295, wherein the viral vector is an adenovirus-derived viral vector or herpes simplex derived viral vector.

297. The method of claim 174, wherein the medicant is an oligonuceotide or nucleotide analog.
